(19) United States
(12) Patent Application Publication
(10) Pub. No.: US 2025/0145957 A1
(43) Pub. Date: May 8, 2025

(54) GENERATION OF DEOXYADENOSINE TRIPHOSPHATE DONOR CELLS AND USES THEREOF

(71) Applicant: UNIVERSITY OF WASHINGTON, Seattle, WA (US)

(72) Inventors: Michael REGNIER, Seattle, WA (US); Charles E. MURRY, Seattle, WA (US); Stephen D. HAUSCHKA, Seattle, WA (US)

(73) Assignee: UNIVERSITY OF WASHINGTON, Seattle, WA (US)

(21) Appl. No.: 18/807,485

(22) Filed: Aug. 16, 2024

Related U.S. Application Data
(63) Continuation-in-part of application No. PCT/US2023/062377, filed on Feb. 10, 2023.
(60) Provisional application No. 63/311,737, filed on Feb. 18, 2022.

(51) Int. Cl. C12N 5/077; C12N 9/02; C12N 15/85

(52) U.S. Cl. CPC C12N 5/0657; C12N 9/0093; C12N 15/85; C12N 2506/45; C12N 2830/15; C12Y 117/04001

(57) ABSTRACT

Provided herein are compositions and methods directed toward the discovery of improved methods for generating deoxyATP (dATP) in cells that can be delivered to a graft site in the heart to enhance cardiac function.

### CROSS REFERENCE TO RELATED APPLICATIONS

This application is a continuation-in-part under 35 U.S.C. § 120 of International Application No. PCT/US2023/062377, filed Feb. 10, 2023, which designates the U.S., and claims benefit under 35 U.S.C. § 119(e) to U.S. Provisional Application No. 63/311,737 filed Feb. 18, 2022, the contents of each of which are incorporated herein by reference in their entireties.

### GOVERNMENT SUPPORT

This invention was made with government support under Grant Nos. R01 HL111197 and R01 HL128368, awarded by the National Institutes of Health. The government has certain rights in the invention.

### SEQUENCE LISTING

The instant application contains a Sequence Listing that has been submitted in XML format via Patent Center and is hereby incorporated by reference in its entirety. Said XML copy, created on Feb. 10, 2023, is named “034186-191480WOPT_SL.xml” and is 39,678 bytes in size.

### TECHNICAL FIELD

The methods and compositions described herein are related to the field of cardiac tissue regeneration.

### BACKGROUND

Positive inotropic agents are used to augment contractility in the treatment of cardiac diseases or disorders that benefit from increasing the force of contraction, including congestive heart failure, cardiomyopathy and in some cases, following myocardial infarction. Traditional inotropic agents work by increasing intracellular cyclic adenosine monophosphate (cAMP) but have not been shown to improve patient survival. Complications of this approach include increased myocardial oxygen consumption, increased arrhythmia burden and activation of adverse signaling pathways such as cell death.

### SUMMARY

The methods and compositions described herein are based, in part, on the discovery of improved methods for generating deoxyATP (dATP) in cells, including cardiomyocytes, that can be delivered to a graft site in the heart to enhance cardiac function. dATP increases the number of myosin S1 heads available for contractions and positions them in an activated state near thin filament actin in sarcomeres. A method for increasing the level of dATP in cardiomyocytes is to overexpress the enzyme ribonucleotide reductase (RNR), which catalyzes the rate-limiting step in de novo dNTP biosynthesis. Gene therapy approaches to overexpress RNR, which increase intracellular dATP, have been shown to recover post-myocardial infarction cardiac function in rodent and pig models.

Provided herein are improved methods for expressing ribonucleotide reductase in cells and using such modified cells as dATP donors to cardiac tissues of a recipient in need of increased contractility to improve cardiac function.

Accordingly, in one aspect as described herein is a cell comprising a nucleic acid sequence encoding a ribonucleotide reductase expression cassette operably linked to a muscle-specific promoter.

In one embodiment of this aspect and all other aspects described herein, the cell is an induced pluripotent stem cell or an embryonic stem cell.

In another embodiment of this aspect and all other aspects described herein, the cell is capable of forming gap junctions with a cardiomyocyte.

In another embodiment of this aspect and all other aspects described herein, the cell capable of forming gap junctions with a cardiomyocyte comprises a cardiomyocyte, a cardiac progenitor cell, a fibroblast, a mesenchymal cell, a smooth muscle cell, an endothelial cell, or a hematopoietic cell.

In another embodiment of this aspect and all other aspects described herein, the cell is an induced pluripotent stem cell, an embryonic stem cell, a cardiac progenitor cell or a cardiomyocyte.

In another embodiment of this aspect and all other aspects described herein, the cardiac progenitor cell or cardiomyocyte are in vitro-differentiated.

In another embodiment of this aspect and all other aspects described herein, the cell is a human cell.

In another embodiment of this aspect and all other aspects described herein, the ribonucleotide reductase expression cassette comprises sequence encoding a ribonucleotide reductase catalytic subunit (RRM1) and a ribonucleotide reductase regulatory subunit (RRM2).

In another embodiment of this aspect and all other aspects described herein, the RRM2 subunit comprises a double mutation and has increased stability compared to the wild-type RRM2 subunit.

In another embodiment of this aspect and all other aspects described herein, the double mutation comprises 30AAA/49ARA mutations in SEQ ID NO. 26.

In another embodiment of this aspect and all other aspects described herein, the sequence of the RRM1 subunit is SEQ ID NO: 1 and the sequence of the RRM2 subunit comprises SEQ ID NO: 3.

In another embodiment of this aspect and all other aspects described herein, the muscle-specific promoter is CK8m or CK8e.

In another embodiment of this aspect and all other aspects described herein, the promoter is cardiac muscle-specific.

In another embodiment of this aspect and all other aspects described herein, the cardiac muscle-specific promoter is selected from MSEC-320, MSEC-455c, MSEC-455a, MSEC-571, MSEC-725a, MSEC-875, and CK8m.

In another embodiment of this aspect and all other aspects described herein, the cardiac-specific promoter is CK8m.

In another aspect as described herein is a cell comprising a nucleic acid sequence encoding a ribonucleotide reductase expression cassette operably linked to a constitutively active promoter.

In one embodiment of this aspect and all other aspects described herein, the constitutively active promoter is selected from CMV, Thymidine Kinase, SV40, EF1A, and CAG promoters.

In another embodiment of this aspect and all other aspects described herein, the cell is capable of forming gap junctions with a cardiomyocyte.

In another embodiment of this aspect and all other aspects described herein, the cell capable of forming gap junctions with a cardiomyocyte comprises a cardiomyocyte, a cardiac progenitor cell, a fibroblast, a mesenchymal cell, a smooth muscle cell, an endothelial cell, or a hematopoietic cell.

In another embodiment of this aspect and all other aspects described herein, the cell is a cardiac progenitor cell or a cardiomyocyte.

In another embodiment of this aspect and all other aspects described herein, the cardiac progenitor cell or cardiomyocyte are in vitro-differentiated.

In another embodiment of this aspect and all other aspects described herein, the cell is a human cell.

In another embodiment of this aspect and all other aspects described herein, the ribonucleotide reductase expression cassette comprises sequence encoding a ribonucleotide reductase catalytic subunit (RRM1) and a ribonucleotide reductase regulatory subunit (RRM2).

In another embodiment of this aspect and all other aspects described herein, the RRM2 subunit comprises a double mutation and has increased stability compared to the wild-type RRM2 subunit.

In another embodiment of this aspect and all other aspects described herein, the double mutation comprises 30AAA/49ARA mutations in SEQ ID NO. 26.

In another embodiment of this aspect and all other aspects described herein, the sequence of the RRM1 subunit is SEQ ID NO: 1 and the sequence of the RRM2 subunit comprises SEQ ID NO: 3.

In another aspect as described herein is a dATP donor cell comprising: a cell of any of the embodiments as described herein formulated for in vivo transplantation.

In another aspect, described herein is a cardiac tissue comprising: a dATP donor cell as described herein, and at least one cardiomyocyte, wherein the dATP donor cell and the at least one cardiomyocyte are connected by one or more gap junctions, thereby permitting dATP to move from the dATP donor cell into the cardiomyocyte.

In one embodiment of this aspect and all other aspects described herein, the dATP donor cell and the at least one cardiomyocyte are in vivo.

In another embodiment of this aspect and all other aspects described herein, the dATP donor cell and the at least one cardiomyocyte comprise a cardiac tissue graft.

In another embodiment of this aspect and all other aspects described herein, the dATP donor cell does not contract or contribute directly to contractility.

In another embodiment of this aspect and all other aspects described herein, the dATP donor cell is a cardiac progenitor cell or a cardiomyocyte.

In another aspect, described herein is a method for treating a cardiac disease or disorder, the method comprising: (i) administering a dATP donor cell as described herein to a graft site in cardiac tissue of a recipient in need thereof, (ii) permitting the dATP donor cell to form gap junctions with at least one cardiomyocyte at the graft site, thereby permitting dATP to move from the dATP donor cell into the cardiomyocyte, and wherein cardiac function in the recipient is improved, thereby treating the cardiac disease or disorder.

In another embodiment of this aspect and all other aspects described herein, the cardiac disease or disorder comprises impaired contractility.

In another embodiment of this aspect and all other aspects described herein, the improvement in cardiac function comprises an increase in regional wall motion, radial or longitudinal strain, fractional shortening, or ejection fraction.

In another embodiment of this aspect and all other aspects described herein, the cardiac disease or disorder comprises a myocardial infarction, an ischemia/reperfusion injury, chronic ischemic heart disease, a cardiomyopathy, drug-induced heart disease, valvular heart disease, congenital heart disease, inflammatory heart disease, a parasitic infection, or heart failure.

In another embodiment of this aspect and all other aspects described herein, the parasitic infection is Chagas Disease.

In another aspect, described herein is a nucleic acid expression construct comprising a nucleic acid sequence encoding a ribonucleotide reductase expression cassette operably linked to a muscle specific or constitutively active promoter.

In another embodiment of this aspect and all other aspects described herein, the ribonucleotide reductase expression cassette comprises sequence encoding a ribonucleotide reductase catalytic subunit (RRM1) and a ribonucleotide reductase regulatory subunit (RRM2).

In another embodiment of this aspect and all other aspects described herein, the RRM2 subunit comprises a double mutation and has increased stability compared to the wild-type RRM2 subunit.

In another embodiment of this aspect and all other aspects described herein, the double mutation comprises 30AAA/49ARA mutations in SEQ ID NO. 26.

In another embodiment of this aspect and all other aspects described herein, the sequence of the RRM1 subunit is SEQ ID NO: 1 and the sequence of the RRM2 subunit comprises SEQ ID NO: 3.

In another embodiment of this aspect and all other aspects described herein, the muscle-specific promoter is CK8m or CK8e.

In another embodiment of this aspect and all other aspects described herein, the promoter is cardiac muscle-specific.

In another embodiment of this aspect and all other aspects described herein, the cardiac muscle-specific promoter is selected from MSEC-320, MSEC-455c, MSEC-455a, MSEC-571, MSEC-725a, MSEC-875, and CK8m.

In another embodiment of this aspect and all other aspects described herein, the cardiac-specific promoter is CK8m.

In another embodiment of this aspect and all other aspects described herein, the constitutively active promoter is selected from CMV, Thymidine Kinase, SV40, EF1A, and CAG promoters.

In another aspect as described herein is a gene therapy vector comprising: a nucleic acid comprising the nucleic acid expression construct of any one of the embodiments as described herein.

In another embodiment of this aspect and all other aspects described herein, the vector comprises an adeno-associated virus (AAV) vector.

In another aspect, described herein is a dATP donor cell comprising the gene therapy vector or the nucleic acid expression construct of any one of the embodiments as described herein.

In another embodiment of this aspect and all other aspects described herein, the cell is an induced pluripotent stem cell or an embryonic stem cell.

In another embodiment of this aspect and all other aspects described herein, the induced pluripotent stem cell is an autologous iPS cell.

In another embodiment of this aspect and all other aspects described herein, the stem cell is grown in-vitro.

In another embodiment of this aspect and all other aspects described herein, the cell is capable of forming gap junctions with a cardiomyocyte.

In another embodiment of this aspect and all other aspects described herein, the cell capable of forming gap junctions with a cardiomyocyte comprises a cardiomyocyte, a cardiac progenitor cell, a fibroblast, a mesenchymal cell, a smooth muscle cell, an endothelial cell, or a hematopoietic cell.

In another embodiment of this aspect and all other aspects described herein, the cell is a cardiac progenitor cell or a cardiomyocyte.

In another embodiment of this aspect and all other aspects described herein, the cardiac progenitor cell or cardiomyocyte are in vitro-differentiated.

In another embodiment of this aspect and all other aspects described herein, the cell is a human cell.

In another aspect, described herein is a method for increasing dATP in a cardiomyocyte or cardiac tissue, the method comprising: (i) contacting a cardiomyocyte with a dATP donor cell of any one of the embodiments as described herein, and (ii) permitting the dATP donor cell to form gap junctions with the cardiomyocyte, thereby permitting dATP to move from the dATP donor cell into the cardiomyocyte, and increasing dATP in the cardiomyocyte or cardiac tissue comprising the cardiomyocyte.

In another embodiment of this aspect and all other aspects described herein, contractility of the contacted cardiomyocyte or cardiac tissue comprising the contacted cardiomyocyte is increased.

In another aspect, described herein is a composition comprising a nucleic acid expression construct comprising a nucleic acid sequence encoding a ribonucleotide reductase expression cassette operably linked to a muscle-specific promoter or a constitutively active promoter.

In another embodiment of this aspect and all other aspects described herein, the ribonucleotide reductase expression cassette comprises sequence encoding a ribonucleotide reductase catalytic subunit (RRM1) and a ribonucleotide reductase regulatory subunit (RRM2).

In another embodiment of this aspect and all other aspects described herein, the RRM2 subunit comprises a double mutation and has increased stability compared to the wild-type RRM2 subunit.

In another embodiment of this aspect and all other aspects described herein, the double mutation comprises 30AAA/49ARA mutations in SEQ ID NO. 26.

In another embodiment of this aspect and all other aspects described herein, the sequence of the RRM1 subunit is SEQ ID NO: 1 and the sequence of the RRM2 subunit comprises SEQ ID NO: 3.

In another embodiment of this aspect and all other aspects described herein, the muscle-specific promoter is CK8m or CK8e.

In another embodiment of this aspect and all other aspects described herein, the promoter is cardiac muscle-specific.

In another embodiment of this aspect and all other aspects described herein, the cardiac muscle-specific promoter is selected from MSEC-320, MSEC-455c, MSEC-455a, MSEC-571, MSEC-725a, MSEC-875, and CK8m.

In another embodiment of this aspect and all other aspects described herein, the cardiac-specific promoter is CK8m.

In another embodiment of this aspect and all other aspects described herein, the constitutively active promoter is selected from CMV, Thymidine Kinase, SV40, EF1A, and CAG promoters.

In another aspect, described herein is a composition of any one of the embodiments as described herein for use as a medicament.

In another aspect, described herein is a composition of any one of the embodiments as described herein for use in the treatment of a myocardial infarction, an ischemia/reperfusion injury, chronic ischemic heart disease, a cardiomyopathy, drug-induced heart disease, valvular heart disease, congenital heart disease, inflammatory heart disease, a parasitic infection, or heart failure.

In another embodiment of this aspect and all other aspects described herein, the parasitic infection is Chagas Disease.

### BRIEF DESCRIPTION OF THE DRAWINGS

FIGS. 1A-1B. Schematic drawings depicting the application of engineered human induced pluripotent stem cell derived cardiomyocytes (hiPSC-CMs) to improve cardiac function in myocardial function (FIG. 1A), and an exemplary mechanism of deoxyATP (dATP) altering myosin structure and activity (FIG. 1B) via (i) SRX to DRX conversion, (ii) increased electrostatic actin-myosin interactions and (iii) increased crossbridge cycling.

FIGS. 2A-2B. dATP diffuses via gap junction to neighboring cardiomyocytes. Schematic illustrating dATP diffusion via gap junctions (FIG. 2A). Micrograph illustrating wild-type cardiomyocytes (WT-CMs; grey) coupled with adenovirally transduced ribonucleotide reductase expressing cardiomyocytes (RNR-CMs; green) (FIG. 2B). Shortening traces illustrating improved contractility of WT-CMs coupled with RNR-CMs.

FIGS. 3A-3B. Elevation of ribonucleotide reductase (RNR) expression in cardiomyocytes by directed mutagenesis of R2 subunit. Down-regulation of R2 subunit during cardiac differentiation (FIG. 3A). Site-directed mutagenesis (FIG. 3B, left) generated R2 mutants (FIG. 3B, right) shows mutant R2 expression levels (72 hours post-transduction) relative to wildtype (WT) R2 for 3 different batches of hiPSC-CMs (2 samples per cell batch).

FIG. 4. Screening of promoters. Human pluripotent stem cell derived cardiomyocytes were transduced with AAV6 at differentiation day 30 for 10 days at MOI of E6 viral particle per cells. The cells were collected 10 days post-transduction for R2 protein expression and dATP levels by mass spectrometry.

FIG. 5. Schematic of exemplary expression cassettes for preclinical experimentation. CAG: chicken beta-actin promoter, CMV enhancer and rabbit beta-globin splice acceptor site. CK8m promoter: differences are in the following transcription binding factor binding sites in CK8 promoter (CArG+MEF2+2GATA+2R Ebox).

FIG. 6. Schematic showing an exemplary method for generation of gene-edited cell lines, clone screening, cardiac differentiation, and dATP clone selection. Cas9=Streptococcus pyogenes Cas9, sgRNA=single-guide RNA.

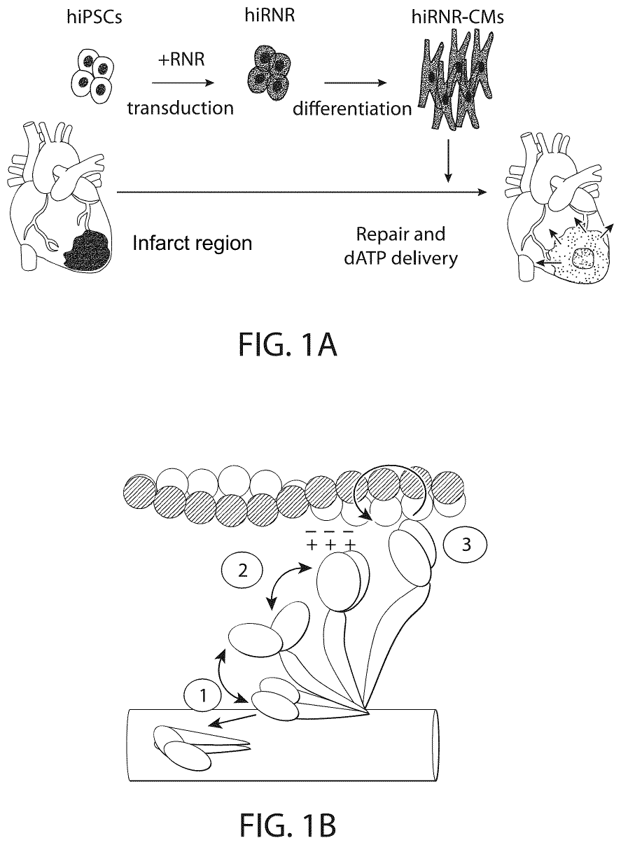

### DETAILED DESCRIPTION

Provided herein are methods and compositions comprising nucleic acid constructs encoding ribonucleotide reductase operably linked to a muscle specific promoter. Such constructs can be used in the generation of cells expressing elevated ribonucleotide reductase activity that have increased levels of deoxyATP (dATP). These cells can be used in methods relating to cardiac regeneration or can be administered to the heart as dATP donor cells to increase contractility in subjects in need thereof.

### Definitions

As used herein, the term “a recipient in need thereof” refers to a subject having a cardiovascular disease or disorder comprising impaired contractility. In some embodiments, the recipient in need thereof can also benefit from a cardiac graft comprising cardiac progenitor cells or in vitro-differentiated cardiomyocytes. For example, the recipient in need thereof can comprise a cardiovascular disease or disorder, including an injury, such as: a myocardial infarction, ischemia/reperfusion injury, cardiomyopathy, muscular dystrophy-associated cardiomyopathies (e.g., Duchenne's muscular dystrophy (DMD)), post-surgical cardiac repair, or heart failure.

As used herein, a “dATP donor cell” is a genetically-modified cell that produces dATP through the activity of a modified or exogenous ribonucleotide reductase (RNR) gene cassette, and which can form gap junctions with a cardiomyocyte. In some embodiments, the expression of one or both RNR subunits in a dATP donor cell is driven by a muscle-specific regulatory element, e.g., including but not limited to a CK8m promoter. A dATP donor cell can be any cell modified to express RNR activity and that has the capacity to form gap junctions with a cardiomyocyte in vivo. In some embodiments, a dATP donor cell is an in vitro-differentiated cardiac progenitor cell as that term is used herein, or an in vitro-differentiated cardiomyocyte.

The term “differentiate”, or “differentiating” is a relative term that indicates a “differentiated cell” is a cell that has progressed further down the developmental pathway than its precursor cell. Thus in some embodiments, a stem cell as the term is defined herein, can differentiate to lineage-restricted precursor cells (e.g., a human cardiac progenitor cell or mid-primitive streak cardiogenic mesoderm progenitor cell), which in turn can differentiate into other types of precursor cells further down the pathway (such as a tissue specific precursor, such as a cardiomyocyte progenitor cell), and then to an end-stage differentiated cell (e.g., a cardiomyocyte), which plays a characteristic role in a certain tissue type, and may or may not retain the capacity to proliferate further.

The term “pluripotent” or “pluripotent stem cell (PSC)” as used herein refers to a cell with the capacity, under different conditions, to differentiate to cell types characteristic of all three germ cell layers (endoderm, mesoderm and ectoderm). Pluripotent cells are characterized primarily by their ability to differentiate to all three germ layers, using, for example, a nude mouse and teratoma formation assay. Pluripotency is also evidenced by the expression of embryonic stem (ES) cell markers, although the preferred test for pluripotency is the demonstration of the capacity to differentiate into cells of each of the three germ layers.

As used herein, the terms “induced pluripotent stem cell,” “iPSC,” “hPSC,” and “human pluripotent stem cell” are used interchangeably herein and refer to a pluripotent cell artificially derived from a differentiated somatic cell (e.g., by reprogramming using one or more methods known in the art). iPSCs are capable of self-renewal and differentiation into cell fate-committed stem cells, including cells of the cardiac lineages, as well as various types of mature cells.

As used herein, “in vitro-differentiated cardiomyocytes” refers to cardiomyocytes that are generated in culture, typically, but not necessarily via step-wise differentiation from a precursor cell such as a human embryonic stem cell, an induced pluripotent stem cell, an early mesoderm cell, a lateral plate mesoderm cell or a cardiac progenitor cell. Thus, while cardiomyocytes in vivo are ultimately derived from a stem cell, i.e., during development of a tissue or organism, a stem cell-derived cardiomyocyte as described herein has been created by in vitro differentiation from a stem cell. As used herein, a cell differentiated in vitro from a stem cell, e.g., an induced pluripotent stem (iPS) cell or embryonic stem cell (“ES cell” or “ESC”), is a “stem-cell derived cardiomyocyte” or “in vitro-differentiated cardiomyocyte” if it has expression of cardiac troponin T (cTnT). Methods for differentiating stem cells in vitro to cardiomyocytes are known in the art and described elsewhere herein. In one embodiment, the cardiomyocytes are differentiated from pluripotent stem cells (e.g., PSC-CMs).

As used herein, the term “cardiomyocyte” refers to a cardiac muscle cell. Cardiomyocytes generally comprise phenotypic and/or structural features associated with cardiac muscle (e.g., electrical phenotypes, sarcomeres, actin, myosin and cardiac troponin T expression, etc.). Typically, cardiomyocytes are terminally differentiated.

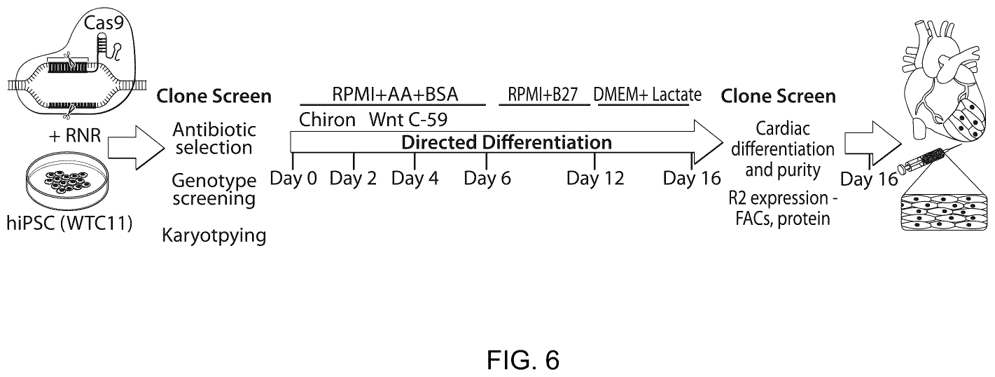

As used herein, the term “cardiac progenitor cell” refers to a cell that is committed to the cardiac lineage but is not a fully differentiated cardiomyocyte. A cardiac progenitor cell can be differentiated in vivo to a cardiomyocyte within the cardiac graft. In some embodiments, the “cardiac progenitor cell” is a cell that is partially, but not fully, differentiated in vitro along the cardiac lineage. In one embodiment, the cardiac progenitor cell for use with the methods and compositions described herein refers to a partially in vitro-differentiated cardiomyocyte that is paused in the differentiation process following inhibition of the Wnt pathway (see e.g., FIG. 6). In another embodiment, the cardiac progenitor cell comprises a cell at day 5 or later (but prior to terminal differentiation of a cardiomyocyte is reached) of an in vitro-differentiation protocol for generating cardiomyocytes.

The term “isolated cell” as used herein refers to a cell that has been removed from an organism in which it was originally found, or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “substantially pure,” with respect to a particular cell population, refers to a population of cells that is at least about 75%, preferably at least about 85%, more preferably at least about 90%, and most preferably at least about 95% pure, with respect to the cells making up a total cell population. That is, the terms “substantially pure” or “essentially purified,” with regard to a population of cardiomyocytes, refers to a population of cells that contains fewer than about 20%, more preferably fewer than about 15%, 10%, 8%, 7%, most preferably fewer than about 5%, 4%, 3%, 2%, 1%, or less than 1%, of cells that are not cardiomyocytes, respectively.

The term “marker” as used herein is used to describe a characteristic and/or phenotype of a cell. Markers can be used, for example, for selection of cells comprising characteristics of interest and can vary with specific cells. Markers are characteristics, whether morphological, structural, functional or biochemical (enzymatic) characteristics of the cell of a particular cell type, or molecules expressed by the cell type. In one aspect, such markers are proteins. Such proteins can possess an epitope for antibodies or other binding molecules available in the art. However, a marker can consist of any molecule found in or on a cell, including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers can be detected by any method available to one of skill in the art. Markers can also be the absence of a morphological characteristic or absence of proteins, lipids etc. Markers can be a combination of a panel of unique characteristics of the presence and/or absence of polypeptides and other morphological or structural characteristics. In one embodiment, the marker is a cell surface marker.

The term “derived from,” used in reference to a stem cell means the stem cell was generated by reprogramming of a differentiated cell to a stem cell phenotype. The term “derived from,” used in reference to a differentiated cell means the cell is the result of differentiation, e.g., in vitro differentiation, of a stem cell. As used herein, “iPSC-CMs” or “induced pluripotent stem cell-derived cardiomyocytes” are used interchangeably to refer to cardiomyocytes derived from an induced pluripotent stem cell. Similarly, “PSC-CMs” or “pluripotent stem cell-derived cardiomyocytes” are used interchangeably to refer to cardiomyocytes derived from a pluripotent stem cell. In some embodiments, the terms “hPSC-CM” or “human pluripotent stem cell derived cardiomyocytes” are used interchangeably to refer to cardiomyocytes derived from a human pluripotent stem cell.

As used herein, the term “fractional shortening” refers to a measure of cardiac muscular contractility determined by assessing the reduction of the length of the end-diastolic diameter that occurs by the end of systole. In the human, a measure of fractional shortening of less than 28% typically is considered impaired. An increase in fractional shortening can be an increase of at least 5%, 10%, 15%, 20%, 25%, 30%, 35%, 40%, 45%, 50%, 55%, 60%, 65%, 70%, 75% or more. In commonly used animal models such as the rat, fractional shortening of less than 40% is typically considered impaired.

As used herein, the term “contractility of the contacted cardiomyocyte or cardiac tissue” refers to the measurement of force of contraction of cardiomyocytes at the cell or tissue level. Exemplary measures for cells and tissues include, but are not limited to, sarcomere shortening, speckle tracking, atomic force microscopy, traction force microscopy, or by measurement of the rate and extent of force development. In intact hearts, contractility can be measured using echocardiography or magnetic resonance imaging (ejection fraction or fractional shortening; radial or longitudinal strain), hemodynamically as developed pressure or preload-recruitable stroke work, or by various biophysical approaches that measure tissue displacement during the cardiac cycle, e.g. wall thickening or percent systolic shortening.

As used herein, the term “ejection fraction” refers to the amount of blood that is pumped out of the left ventricle with each contraction.

As used herein, the term “contacting” when used in reference to a cell, encompasses introducing an agent, surface, scaffold etc. to the cell in a manner that permits physical contact of the cell with the agent, surface, scaffold etc.

As used herein, the term, “cardiac disease” refers to a disease that affects the cardiac tissue of a subject. Non-limiting examples of cardiac diseases include cardiomyopathy, cardiac arrhythmias, myocardial infarction, chronic ischemic heart disease, metabolic heart disease, inflammatory heart disease, heart failure, cardiac hypertrophy, long QT syndrome, arrhythmogenic right ventricular dysplasia (ARVD), catecholaminergic polymorphic ventricular tachycardia (CPVT), Barth syndrome, congenital defects, Chagas disease, and Duchenne muscular dystrophy.

The terms “patient”, “subject” and “individual” are used interchangeably herein, and refer to an animal, particularly a human, to whom treatment, including prophylactic treatment is provided. The term “subject” as used herein refers to human and non-human animals. The term “non-human mammals” includes all mammals, such as non-human primates, (particularly higher primates), sheep, dog, rodent (e.g. mouse or rat), guinea pig, goat, pig, cat, rabbits, cows, etc. In one embodiment of any of the aspects, the subject is human. In another embodiment, of any of the aspects, the subject is an experimental animal or animal substitute as a disease model. In another embodiment, of any of the aspects, the subject is a domesticated animal including companion animals (e.g., dogs, cats, rats, guinea pigs, hamsters etc.). A subject can have previously received a treatment for a disease, or has never received treatment for a disease. A subject can have previously been diagnosed with having a disease, or has never been diagnosed with a disease. A subject can be of any age including, e.g., a fetus, a neonate, a toddler, a child, an adolescent, an adult, a geriatric subject etc.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased,” “increase,” “increases,” or “enhance” or “activate” are all used herein to generally mean an increase of a property, level, or other parameter by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Example devices, methods, and systems are described herein. It should be understood the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood other embodiments may include more or less of each element shown in a given Figure. Further, some of the illustrated elements may be combined or omitted. Yet further, an example embodiment may include elements not illustrated in the Figures. As used herein, with respect to measurements, “about” means +/−5%.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

It will be appreciated that, although specific embodiments of the invention have been described herein for purposes of illustration, various modifications may be made without deviating from the spirit and scope of the invention. Accordingly, the invention is not limited except as by the claims.

### Cardiovascular Diseases

The methods and compositions described herein relate to methods and compositions for enhancing muscle contractility, e.g., cardiac muscle contractility, in vivo in a subject in need thereof. The methods described herein can be used to treat, ameliorate, prevent or slow the progression of a number of diseases or their symptoms, such as those resulting in pathological damage to the structure and/or function of the heart (e.g., remodeling in response to heart failure).

A cardiovascular disease is a disease that affects the heart and/or circulatory system of a subject. Such cardiac diseases or cardiac-related disease include, but are not limited to, myocardial infarction, cardiac arrhythmia, heart failure, atherosclerotic heart disease, cardiomyopathy, congenital heart defect (e.g., non-compaction cardiomyopathy, septal defects, hypoplastic left heart), hypertrophic cardiomyopathy, dilated cardiomyopathy, cardiac hypertrophy, myocarditis, arrhythmogenic right ventricular dysplasia (ARVD), long QT syndrome, catecholaminergic polymorphic ventricular tachycardia (CPVT), Barth syndrome, valvular stenosis, regurgitation, ischemia, fibrillation, polymorphic ventricular tachycardia, and muscular dystrophies such as Duchenne or related cardiac disease, and cardiomegaly. Chagas Disease is an insect borne disease by infection with Trypanosoma cruzi and it causes cardiac disease including rhythmic abnormalities and dilation of the cardiac muscle. Generally, the methods and compositions described herein will be most beneficial for the treatment of cardiac diseases or disorders with impaired contractility, for example, heart failure, myocardial infarction, and cardiomyopathies.

Symptoms of cardiovascular disease can include but are not limited to syncope, fatigue, shortness of breath, chest pain, lower limb edema, and palpitations. A cardiovascular disease is generally diagnosed by a physical examination, blood tests, and/or an electrocardiogram (EKG). An abnormal EKG is an indication that the subject has an abnormal cardiac rhythm or cardiac arrhythmia.

In some embodiments of any of the aspects, the subject has or is at risk for having a cardiovascular disease or a cardiac event.

dATP Donor Cells

As described herein, “dATP donor cells” are cells modified to express increased levels of ribonucleotide reductase activity, which increases dATP production, wherein the cells are capable of forming gap junctions with cardiomyocytes; dATP can transit such gap junctions and thereby increase contractile function in the cardiomyocytes. It is contemplated that any cell capable of forming gap junctions with a cardiomyocyte can be engineered to be a dATP donor cell. In some embodiments, dATP donor cells include, but are not limited to cardiomyocytes, cardiomyocyte precursors, or progenitors with a ribonucleotide reductase cassette driven by a muscle-specific promoter. The ribonucleotide reductase cassette can include, but not be limited to RRM1 expression elements and RRM2 expression elements, where the dATP donor cells can form gap junctions with cardiomyocytes and thereby deliver dATP to them to improve cardiac contractility and/or function.

Gap junctions are formed when a hemichannel in the plasma membrane of one cell docks with a hemichannel in the plasma membrane of an adjacent cell, with hemichannels made up of six connexin protein subunits. In some embodiments, the presence of one or more of the connexin proteins can be used to form a gap junction including, but not limited to connexin (Cx)31.9, Cx32, Cx37, Cx40, Cx43, and Cx45. Information regarding gap junctions and cells that can form them with cardiomyocytes can be found in Johnson R D, Camelliti P. Role of Non-Myocyte Gap Junctions and Connexin Hemichannels in Cardiovascular Health and Disease: Novel Therapeutic Targets? Int J Mol Sci. 2018 Mar. 15; 19(3):866; Menges L, Krawutschke C, Fuchtbauer E M, et al. Mind the gap (junction): cGMP induced by nitric oxide in cardiac myocytes originates from cardiac fibroblasts. Br J Pharmacol. 2019; 176(24):4696-4707; Talman et al. Cardiomyocyte-Endothelial Cell Interactions in Cardiac Remodeling and Regeneration. Front. Cardiovasc. Med., 26 Jul. 2018, Vol 5; Rodriguez-Sinovas, A. et al. Connexins in the Heart: Regulation, Function and Involvement in Cardiac Disease. Int. J. Mol. Sci. 2021, 22(9), 4413; Hulsmans M, Clauss S, Xiao L, et al. Macrophages Facilitate Electrical Conduction in the Heart. Cell. 2017; 169(3):510-522.e20; Hulsmans M, Clauss S, Xiao L, et al. Macrophages Facilitate Electrical Conduction in the Heart. Cell. 2017; 169(3):510-522.e20. doi:10.1016/j.cell.2017.03.050; and Valiunas, V., Doronin, S., Valiuniene, L., Potapova, I., Zuckerman, J., Walcott, B., Robinson, R. B., Rosen, M. R., Brink, P. R. and Cohen, I. S. (2004), Human mesenchymal stem cells make cardiac connexins and form functional gap junctions. The Journal of Physiology, 555: 617-626, the contents of which are incorporated by reference herein in their entireties. The following describes the various considerations for producing and using dATP donor cells for the methods and compositions disclosed herein.

### Pluripotent Stem Cell Sources

The methods and compositions described herein can generate dATP donor cells, e.g., from cardiomyocytes or other cell types differentiated in vitro, e.g., from embryonic stem cells, pluripotent stem cells, such as induced pluripotent stem cells, or other stem cells that permit such differentiation. The following describes various stem cells that can be used to prepare cardiomyocytes.

Stem cells are cells that retain the ability to renew themselves through mitotic cell division and can differentiate into more specialized cell types. Three broad types of mammalian stem cells include: embryonic stem (ES) cells that are found in blastocysts, induced pluripotent stem cells (iPSCs) that are reprogrammed from somatic cells, and adult stem cells that are found in adult tissues. Other sources of pluripotent stem cells can include amnion-derived or placental-derived stem cells. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

Cardiomyocytes useful in the methods and compositions described herein can be differentiated from both embryonic stem cells and induced pluripotent stem cells, among others. Cardiomyocytes differentiated directly from other cell types, e.g., by transdifferentiation or other methods, are also applicable. In one embodiment, the compositions and methods provided herein use human cardiomyocytes differentiated from embryonic stem cells. Alternatively, in some embodiments, the compositions and methods provided herein do not encompass generation or use of human cardiogenic cells made from cells taken from a viable human embryo.

Embryonic stem cells and methods for their retrieval are well known in the art and are not described in detail herein. A cell has the phenotype of an embryonic stem cell if it possesses one or more of the unique characteristics of an embryonic stem cell such that that cell can be distinguished from other cells. Exemplary distinguishing embryonic stem cell characteristics include, without limitation, morphology, gene expression or marker profile, proliferative capacity, differentiation capacity, responsiveness to particular culture conditions, and the like.

Cells derived from embryonic sources can include embryonic stem cells or stem cell lines obtained from a stem cell bank or other recognized depository institution. Other means of producing stem cell lines include methods comprising the use of a blastomere cell from an early stage embryo prior to formation of the blastocyst (at around the 8-cell stage). Such techniques correspond to the pre-implantation genetic diagnosis technique routinely practiced in assisted reproduction clinics. The single blastomere cell is co-cultured with established ES-cell lines and then separated from them to form fully competent ES cell lines.

Embryonic stem cells are considered to be undifferentiated when they have not committed to a specific differentiation lineage. Such cells display morphological characteristics that distinguish them from differentiated cells of embryo or adult origin. Undifferentiated embryonic stem (ES) cells are easily recognized by those skilled in the art, and typically appear in the two dimensions of a microscopic view in colonies of cells with high nuclear/cytoplasmic ratios and prominent nucleoli. In some embodiments, the human cardiomyocytes described herein are not derived from embryonic stem cells or any other cells of embryonic origin.

Adult stem cells are stem cells derived from tissues of a post-natal or post-neonatal organism or from an adult organism. An adult stem cell is structurally distinct from an embryonic stem cell not only in markers it does or does not express relative to an embryonic stem cell, but also by the presence of epigenetic differences, e.g. differences in DNA methylation patterns.

In some embodiments, the methods and compositions described herein utilize cardiomyocytes that are differentiated in vitro from induced pluripotent stem cells. An advantage of using iPSCs to generate cardiomyocytes or other cells for the compositions described herein is that the cells can be derived from the same subject to which the desired human cardiomyocytes are to be administered. That is, a somatic cell can be obtained from a subject, reprogrammed to an induced pluripotent stem cell, and then re-differentiated into a human cardiomyocyte cell to be administered to the subject (e.g., autologous cells). Since the cardiomyocytes (or their differentiated progeny) are essentially derived from an autologous source, the risk of engraftment rejection or allergic responses is reduced compared to the use of cells from another subject or group of subjects.

In some embodiments, the cardiomyocytes or other cells useful for the compositions described herein are derived from non-autologous or allogeneic sources.

Although differentiation is generally irreversible under physiological contexts, several methods have been developed in recent years to reprogram somatic cells to induced pluripotent stem cells or directly into other differentiated cell types such as cardiomyocytes and neurons. Exemplary methods are known to those of skill in the art and are described briefly herein below.

Reprogramming is a process that alters or reverses the differentiation state of a differentiated cell (e.g., a somatic cell). Stated another way, reprogramming is a process of driving the differentiation of a cell backwards to a more undifferentiated or more primitive type of cell, or laterally, directly into a different cell type (e.g., fibroblast to cardiomyocyte). It should be noted that placing many primary cells in culture can lead to some loss of fully differentiated characteristics. However, simply culturing such cells included in the term differentiated cells does not render these cells non-differentiated cells (e.g., undifferentiated cells) or pluripotent cells. The transition of a differentiated cell to pluripotency requires a reprogramming stimulus beyond the stimuli that lead to partial loss of differentiated character when differentiated cells are placed in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. In some embodiments, reprogramming encompasses complete reversion of the differentiation state of a differentiated cell (e.g., a somatic cell) to a pluripotent state or a multipotent state. In some embodiments, reprogramming encompasses complete or partial reversion of the differentiation state of a differentiated cell (e.g., a somatic cell) to an undifferentiated cell (e.g., an embryonic-like cell). Reprogramming can result in expression of particular genes by the cells, the expression of which further contributes to reprogramming. In certain embodiments described herein, reprogramming of a differentiated cell (e.g., a somatic cell) causes the differentiated cell to assume an undifferentiated state with the capacity for self-renewal and differentiation to cells of all three germ cell lineages. The resulting cells are referred to as “reprogrammed cells,” or “induced pluripotent stem cells (iPSCs or iPS cells), or if directly reprogrammed into another fate, induced cardiomyocytes or neurons (iCardiomyocytes or iNeurons, respectively).”

The specific approach or method used to generate pluripotent stem cells from somatic cells (e.g., any cell of the body with the exclusion of a germ line cell; fibroblasts etc.) is not critical to the claimed invention. Thus, any method that re-programs a somatic cell to the pluripotent phenotype would be appropriate for use in the methods described herein. Examples use virally-introduced reprogramming factors or chemically-induced reprogramming—see, e.g., EP 1970446, US2009/0047263, US2009/0068742, and 2009/0227032, which are incorporated herein in their entirety by reference. Efficient RNA-mediated reprogramming has also been described (see, e.g., Kogut et al., Nature Commun. 9: 745 (2018).

iPS cells can be generated or derived from terminally differentiated somatic cells, as well as from adult stem cells, or somatic stem cells. That is, a non-pluripotent progenitor cell can be rendered pluripotent or multipotent by reprogramming.

The efficiency of reprogramming (i.e., the number of reprogrammed cells) derived from a population of starting cells can be enhanced by the addition of various small molecules as shown by Shi, Y., et al (2008) Cell-Stem Cell 2:525-528, Huangfu, D., et al (2008) Nature Biotechnology 26(7):795-797, and Marson, A., et al (2008) Cell-Stem Cell 3:132-135. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg 1, Eras, Gdf3, Fgf4, Cripto, Dax1, Zpf296, Slc2a3, Rex1, Utf1, and Nat1. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

Reprogrammed somatic cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; β-III-tubulin; α-smooth muscle actin (α-SMA); fibroblast growth factor 4 (Fgf4), Cripto, Dax1; zinc finger protein 296 (Zfp296); N-acetyltransferase-1 (Nat1); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fthl17; Sa114; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tell); DPPA3/Stella; DPPA4; other general markers for pluripotency, etc. Other markers can include Dnmt3L; Sox15; Stat3; Grb2; β-catenin, and Bmi1. Such cells can also be characterized by the down-regulation of markers characteristic of the somatic cell from which the induced pluripotent stem cell is derived.

### In Vitro Differentiation of Cardiomyocytes

The methods and compositions described herein can generate dATP donor cells that are in vitro-differentiated cardiomyocytes. Methods for the differentiation of cardiomyocytes from ESCs or IPSCs are known in the art. See, e.g., LaFlamme et al., Nature Biotech 25:1015-1024 (2007), which describes the differentiation of cardiomyocytes. These approaches use various factors and conditions to activate and guide differentiation programs leading to their respective cell types. Pathways and certain of the factors involved in them are discussed in the following.

In certain embodiments, the step-wise differentiation of ESCs or iPSCs to cardiomyocytes proceeds in the following order: ESC or iPSC>cardiogenic mesoderm>cardiac progenitor cells>cardiomyocytes (see e.g., Lian et al. Nat Prot (2013); US Applicant No. 2017/0058263 A1; 2008/0089874 A1; 2006/0040389 A1; U.S. Pat. Nos. 10,155,927; 9,994,812; 9,663,764, US 20170240861, the contents of each of which are incorporated herein by reference in their entireties).

As will be appreciated by those of skill in the art, in vitro-differentiation of cardiomyocytes produces an end-result of a cell having the phenotypic and morphological features of a cardiomyocyte but that the differentiation steps of in vitro-differentiation need not be the same as the differentiation that occurs naturally in the embryo. That is, during in vitro differentiation to a cardiomyocyte, it is specifically contemplated herein that the step-wise differentiation approach utilized to produce such cells need not proceed through every progenitor cell type that has been identified during embryogenesis and can essentially “skip” over certain stages of development that occur during embryogenesis.

Exemplary methods for generating cardiomyocytes or cardiac progenitor cells from pluripotent stem cells (e.g., embryonic stem cells or induced pluripotent stem cells) are described in e.g., US2020-0085880, the contents of which are incorporated herein by reference in their entirety.

In an alternative approach, cardiac progenitor cells or cardiomyocytes can be generated by direct reprogramming or transdifferentiation of an adult somatic cell.

In some embodiments, the dATP donor cells are not cardiomyocytes, but have the capacity to form gap junctions with cardiomyocytes. In some embodiments, the methods and compositions described herein utilize cardiomyocytes that are differentiated in vitro from induced pluripotent stem cells. An advantage of using iPSCs to generate cardiomyocyte or other cells for the compositions described herein is that the cells can be derived from the same subject to which the desired human cardiomyocytes and/or epicardial cells are to be administered. That is, a somatic cell can be obtained from a subject, reprogrammed to an induced pluripotent stem cell, and then re-differentiated into a human cardiomyocyte or other cell to be administered to the subject (e.g., autologous cells). Since the cardiomyocytes and other cells (or their differentiated progeny) are essentially derived from an autologous source, the risk of engraftment rejection is reduced compared to the use of cells from another subject or group of subjects. In some embodiments, the cardiomyocytes and/or epicardial cells useful for the compositions described herein are derived from non-autologous sources. In addition, the use of iPSCs negates the need for cells obtained from an embryonic source. Thus, in one embodiment, the stem cells used to generate epicardial cells or cardiomyocytes for use in the compositions and methods described herein are not embryonic stem cells. Methods are known in the art for differentiating iPS cells into a wide range of cell types, including cardiomyocytes (see, e.g., LaFlamme et al., Nature Biotech 25:1015-1024 (2007)), macrophages (see, e.g., Lachmann et al., Stem Cell Repts. 4(2): 282-296 (2015)), epicardiomyocytes (see, US Patent publication US 2020-0085880, incorporated by reference in its entirety), fibroblasts (see, e.g., Hewitt, K. J et al. PLoS One. 2011; 6(2): e17128), mesenchymal stem cells (see, e.g., Soontararak et al. Stem Cells Transl Med. 2018 June; 7(6): 456-467), and smooth muscle cells (see, e.g., Maguire et al. Arteriosclerosis, Thrombosis, and Vascular Biology, 2017; 37: 2026-2037).

### Ribonucleotide Reductase

Ribonucleotide reductase (RNR), also known as ribonucleotide diphosphate reductase (rNDP), is an enzyme that catalyzes the reaction of ribonucleotides to deoxyribonucleotides, which are essential components in the synthesis of DNA. RNR is conserved in all living organisms. The RNR enzyme catalyzes the de novo synthesis of dNDPs.

As used herein, “RRM1” or “ribonucleotide reductase catalytic subunit M1” or “an RRM1 construct” refers to the large, catalytic site-containing, subunit of the RNR complex. Sequences for RRM1 are known for a number of species, e.g., human RRM1 (NCBI Gene ID: 6240) mRNA (NCBI Ref Seq: NM 001033.5) and polypeptide (NCBI Ref Seq: NP_001024.1). In some embodiments of any of the aspects described herein, the RRM1 nucleic acid or polypeptide can be an isoform, ortholog, variant, and/or allele of SEQ ID NO: 1 or SEQ ID NO: 2, respectively.

| SEQ ID NO: 1 is the nucleotide sequence encoding Rrml, isoform 1. | |
|---|---|
| (SEQ ID NO: 1) | |
| | atgcat gtgatcaagc gagatggccg |
| 241 | ccaagaacga gtcatgtttg acaaaattac atctcgaatc cagaagcttt gttatggact |
| 301 | caatatggat tttgttgatc ctgctcagat caccatgaaa gtaatccaag gcttgtacag |
| 361 | tggggtcacc acagtggaac tagatacttt ggctgctgaa acagctgcaa ccttgactac |
| 421 | taagcaccct gactatgcta tcctggcagc caggatcgct gtctctaact tgcacaaaga |
| 481 | aacaaagaaa gtgttcagtg atgtgatgga agacctctat aactacataa atccacataa |
| 541 | tggcaaacac tctcccatgg tggccaagtc aacattggat attgttctgg ccaataaaga |
| 601 | tcgcctgaat tctgctatta tctatgaccg agatttctct tacaattact tcggctttaa |
| 661 | gacgctagag cggtcttatt tgttgaagat caatggaaaa gtggctgaaa gaccacaaca |
| 721 | tatgttgatg agagtatctg ttgggatcca caaagaagac attgatgcag caattgaaac |
| 781 | atataatctt ctttctgaga ggtggtttac tcatgcttcg cccactctct tcaatgctgg |
| 841 | taccaaccgc ccacaacttt ctagctgttt tcttctgagt atgaaagatg acagcattga |
| 901 | aggcatttat gacactctaa agcaatgtgc attgatttct aagtctgctg gaggaattgg |
| 961 | tgttgctgtg agttgtattc gggctactgg cagctacatt gctgggacta atggcaattc |
| 1021 | caatggcctt gtaccgatgc tgagagtata taacaacaca gctcgatatg tggatcaagg |
| 1081 | tgggaacaag cgtcctgggg catttgctat ttacctggag ccttggcatt tagacatctt |
| 1141 | tgaattcctt gatttaaaga agaacacagg aaaggaagag cagcgtgcca gagatctttt |
| 1201 | ctttgctctt tggattccgg atctcttcat gaaacgagtg gagactaatc aggactggtc |
| 1261 | tttgatgtgt ccaaatgagt gtcctggtct ggatgaggtt tggggagagg aatttgagaa |
| 1321 | actatatgca agttatgaga aacaaggtcg tgtccgcaaa gttgtaaaag ctcagcagct |
| 1381 | ttggtatgcc atcattgagt ctcagacgga aacaggcacc ccgtatatgc tctacaaaga |
| 1441 | ttcctgtaat cgaaagagca accagcagaa cctgggaacc atcaaatgca gcaacctgtg |
| 1501 | cacagaaata gtggagtaca ccagcaaaga tgaggttgct gtttgtaatt tggcttccct |
| 1561 | ggccctgaat atgtatgtca catcagaaca cacatacgac tttaagaagt tggctgaagt |
| 1621 | cactaaagtc gttgtccgaa acttgaataa aattattgat ataaactact atcctgtacc |
| 1681 | agaggcatgc ctatcaaata aacgccatcg ccccattgga attggggtac aaggtctggc |
| 1741 | agatgctttt atcctgatga gatacccttt tgagagtgca gaagcccagt tactgaataa |
| 1801 | gcagatcttt gaaactattt attatggtgc tctggaagcc agctgtgacc ttgccaagga |
| 1861 | gcagggccca tacgaaacct atgagggctc tccagttagc aaaggaattc ttcagtatga |
| 1921 | tatgtggaat gttactccta cagacctatg ggactggaag gttctcaagg agaagattgc |
| 1981 | aaagtatggt ataagaaaca gtttacttat tgccccgatg cctacagctt ccactgctca |
| 2041 | gatcctgggg aataatgagt ccattgaacc ttacaccagc aacatctata ctcgcagagt |
| 2101 | cttgtcagga gaatttcaga ttgtaaatcc tcacttattg aaagatctta ccgagcgggg |
| 2161 | cctatggcat gaagagatga aaaaccagat tattgcatgc aatggctcta ttcagagcat |
| 2221 | accagaaatt cctgatgacc tgaagcaact ttataaaact gtgtgggaaa tctctcagaa |
| 2281 | aactgttctc aagatggcag ctgagagagg tgctttcatt gatcaaagcc aatctttgaa |
| 2341 | catccacatt gctgagccta actatggcaa actcactagt atgcacttct acggctggaa |
| 2401 | gcagggtttg aagactggga tgtattattt aaggacaaga ccagcggcta atccaatcca |
| 2461 | gttcactcta aataaggaga agctaaaaga taaagaaaag gtatcaaaag aggaagaaga |
| 2521 | gaaggagagg aacacagcag ccatggtgtg ctctttggag aatagagatg aatgtctgat |
| 2581 | gtgtggatcc tga |

SEQ ID NO: 2 is the amino acid sequence for Rrm1, isoform 1.
(SEQ ID NO: 2)
MHVIKRDGRQERVMFDKITSRIQKLCYGLNMDFVDPAQITMKVI
QGLYSGVTTVELDTLAAETAATLTTKHPDYAILAARIAVSNLHKETKKVFSDVMEDLY
NYINPHNGKHSPMVAKSTLDIVLANKDRLNSAIIYDRDFSYNYFGFKTLERSYLLKIN
GKVAERPQHMLMRVSVGIHKEDIDAAIETYNLLSERWFTHASPTLFNAGTNRPQLSSC
FLLSMKDDSIEGIYDTLKQCALISKSAGGIGVAVSCIRATGSYIAGTNGNSNGLVPML
RVYNNTARYVDQGGNKRPGAFAIYLEPWHLDIFEFLDLKKNTGKEEQRARDLFFALWI
PDLFMKRVETNQDWSLMCPNECPGLDEVWGEEFEKLYASYEKQGRVRKVVKAQQLWYA
IIESQTETGTPYMLYKDSCNRKSNQQNLGTIKCSNLCTEIVEYTSKDEVAVCNLASLA
LNMYVTSEHTYDFKKLAEVTKVVVRNLNKIIDINYYPVPEACLSNKRHRPIGIGVQGL
ADAFILMRYPFESAEAQLLNKQIFETIYYGALEASCDLAKEQGPYETYEGSPVSKGIL
QYDMWNVTPTDLWDWKVLKEKIAKYGIRNSLLIAPMPTASTAQILGNNESIEPYTSNI
YTRRVLSGEFQIVNPHLLKDLTERGLWHEEMKNQIIACNGSIQSIPEIPDDLKQLYKT
VWEISQKTVLKMAAERGAFIDQSQSLNIHIAEPNYGKLTSMHFYGWKQGLKTGMYYLR
TRPAANPIQFTLNKEKLKDKEKVSKEEEEKERNTAAMVCSLENRDECLMCGS

As used herein, “RRM2” or “ribonucleotide reductase catalytic subunit M2” or an “RRM2 construct” refers to the small subunit of the RNR complex. Sequences for RRM2 are known for a number of species, e.g., human RRM2 (NCBI Gene ID: 6241) mRNA (NCBI Ref Seq: NM_001034.4) and polypeptide (NCBI Ref Seq: NP_001025.1). In some embodiments, the RRM2 nucleic acid or polypeptide can be an isoform, ortholog, variant, and/or allele of SEQ ID NO: 3 or SEQ ID NO: 4, respectively.

SEQ ID NO: 3 is the nucleic acid sequence for
human RRM2, isoform 2.
(SEQ ID NO: 3)
ATGCTCTCCCTCCGTGTCCCGCTCGCGCCCATCACGGACCCGCAGCAGCT
GCAGCTCTCGCCGCTGAAGGGGCTCAGCTTGGTCGACAAGGAGAACACGC
CGCCGGCCCTGAGCGGGACCCGCGTCCTGGCCAGCAAGACCGCGAGGAGG
ATCTTCCAGGAGCCCACGGAGCCGAAAACTAAAGCAGCTGCCCCCGGCGT
GGAGGATGAGCCGCTGCTGAGAGAAAACCCCCGCCGCTTTGTCATCTTCC
CCATCGAGTACCATGATATCTGGCAGATGTATAAGAAGGCAGAGGCTTCC
TTTTGGACCGCCGAGGAGGTGGACCTCTCCAAGGACATTCAGCACTGGGA
ATCCCTGAAACCCGAGGAGAGATATTTTATATCCCATGTTCTGGCTTTCT
TTGCAGCAAGCGATGGCATAGTAAATGAAAACTTGGTGGAGCGATTTAGC
CAAGAAGTTCAGATTACAGAAGCCCGCTGTTTCTATGGCTTCCAAATTGC
CATGGAAAACATACATTCTGAAATGTATAGTCTTCTTATTGACACTTACA
TAAAAGATCCCAAAGAAAGGGAATTTCTCTTCAATGCCATTGAAACGATG
CCTTGTGTCAAGAAGAAGGCAGACTGGGCCTTGCGCTGGATTGGGGACAA
AGAGGCTACCTATGGTGAACGTGTTGTAGCCTTTGCTGCAGTGGAAGGCA
TTTTCTTTTCCGGTTCTTTTGCGTCGATATTCTGGCTCAAGAAACGAGGA
CTGATGCCTGGCCTCACATTTTCTAATGAACTTATTAGCAGAGATGAGGG
TTTACACTGTGATTTTGCTTGCCTGATGTTCAAACACCTGGTACACAAAC
CATCGGAGGAGAGAGTAAGAGAAATAATTATCAATGCTGTTCGGATAGAA
CAGGAGTTCCTCACTGAGGCCTTGCCTGTGAAGCTCATTGGGATGAATTG
CACTCTAATGAAGCAATACATTGAGTTTGTGGCAGACAGACTTATGCTGG
AACTGGGTTTTAGCAAGGTTTTCAGAGTAGAGAACCCATTTGACTTTATG
GAGAATATTTCACTGGAAGGAAAGACTAACTTCTTTGAGAAGAGAGTAGG
CGAGTATCAGAGGATGGGAGTGATGTCAAGTCCAACAGAGAATTCTTTTA
CCTTGGATGCTGACTTCTAA
SEQ ID NO: 4 is the amino acid sequence for Rrm2,
isoform 2.
(SEQ ID NO: 4)
MLSLRVPLAPITDPQQLQLSPLKGLSLVDKENTPPALSGTRVLASKTARR
IFQEPTEPKTKAAAPGVEDEPLLRENPRRFVIFPIEYHDIWQMYKKAEAS
FWTAEEVDLSKDIQHWESLKPEERYFISHVLAFFAASDGIVNENLVERFS
QEVQITEARCFYGFQIAMENIHSEMYSLLIDTYIKDPKEREFLFNAIETM
PCVKKKADWALRWIGDKEATYGERVVAFAAVEGIFFSGSFASIFWLKKRG
LMPGLTFSNELISRDEGLHCDFACLMFKHLVHKPSEERVREIIINAVRIE
QEFLTEALPVKLIGMNCTLMKQYIEFVADRLMLELGFSKVFRVENPFDFM
ENISLEGKTNFFEKRVGEYQRMGVMSSPTENSFTLDADF

RRM1 and RRM2 proteins as described herein need to be capable of forming an active RNR complex. Brignole et al., eLife 2018; 7:e31502, which is incorporated herein by reference, describes a 3.3A resolution cryo-EM structure of human ribonucleotide reductase complexed with substrate and allosteric regulators (ATP and dATP)—this near-atomic resolution structure illustrates amino acids and structural domains in the two subunits that interact with each other and illustrates domains necessary for allosteric regulation.

In some embodiments of any of the aspects, the RRM2-encoding nucleic acid is linked to the RRM1-encoding nucleic acid, e.g., through a type 2A peptide-encoding sequence, such as P2A. P2A is a non-limiting example of a 2A self-cleaving peptide, which can induce the cleavage of the recombinant protein when expressed in a cell. See, e.g., Kolwicz et al., Molecular Therapy 24: 240-250 (2016), which is incorporated herein by reference in its entirety. Non-limiting examples of 2A self-cleaving peptides include T2A, P2A, E2A, and F2A. Any self-cleaving peptide sequence known in the art can be used to link RRM1 to RRM2.

SEQ ID NO: 5 is an exemplary codon-optimized Rrm2
nucleic acid sequence.
ATGCTGAGTCTGAGGGTCCCACTGGCACCTATCACCGATCCACAGCAGCT
GCAGCTGAGCCCACTGAAAGGCCTGAGTCTGGTCGATAAAGAGAACACAC
CACCTGCACTGAGTGGCACTCGGGTGCTGGCATCAAAGACCGCCCGGAGA
ATTTTCCAGGAGCCAACCGAACCCAAAACAAAGGCCGCTGCACCTGGGGT
CGAGGACGAACCACTGCTGAGAGAGAATCCCAGGCGCTTCGTGATTTTTC
CTATCGAATACCACGATATTTGGCAGATGTATAAGAAAGCTGAGGCAAGT
TTCTGGACAGCTGAGGAAGTGGACCTGAGCAAAGACATCCAGCACTGGGA
ATCCCTGAAGCCAGAGGAAAGGTACTTCATTTCTCATGTGCTGGCATTCT
TTGCCGCTAGTGACGGGATCGTGAACGAGAATCTGGTCGAACGCTTTAGC
CAGGAGGTGCAGATCACTGAAGCCCGATGCTTCTATGGATTTCAGATTGC
TATGGAGAACATCCATTCAGAAATGTACAGCCTGCTGATTGACACCTATA
TCAAAGATCCTAAGGAGCGCGAGTTCCTGTTTAATGCCATTGAGACAATG
CCATGTGTGAAGAAAAAGGCAGACTGGGCTCTGCGATGGATCGGCGATAA
GGAGGCTACTTACGGGGAAAGAGTGGTCGCATTCGCAGCCGTGGAGGGAA
TTTTCTTTTCTGGCAGTTTCGCTTCCATCTTTTGGCTGAAAAAGCGAGGC
CTGATGCCTGGGCTGACCTTTTCCAACGAGCTGATTTCTCGCGACGAAGG
CCTGCACTGCGATTTCGCCTGTCTGATGTTTAAACACCTGGTGCATAAGC
CCTCTGAGGAACGAGTCCGGGAGATCATTATCAACGCAGTGAGGATCGAG
CAGGAGTTCCTGACAGAAGCCCTGCCTGTCAAACTGATTGGCATGAATTG
CACTCTGATGAAGCAGTACATCGAGTTTGTGGCCGACAGGCTGATGCTGG
AACTGGGATTCTCAAAGGTGTTTCGCGTCGAGAACCCATTCGATTTTATG
GAGAATATCAGCCTGGAAGGCAAAACAAACTTCTTTGAGAAGAGAGTCGG
GGAATATCAGAGGATGGGCGTGATGAGCAGCCCCACTGAGAATAGCTTCA
CCCTGGACGCCGATTTTTGA

In one embodiment, the Rrm2 and Rrm1 mRNAs are bicistronic, i.e., on the same vector and/or under the control of the same promoter. In one embodiment, the Rrm2-encoding nucleic acid is linked to Rrm1-encoding nucleic acid, e.g., through sequence encoding a self-cleaving peptide. P2A is a non-limiting example of a 2A self-cleaving peptide, which can induce the cleaving of the recombinant protein in cell. In one embodiment, the Rrm2-encoding nucleic acid is linked to Rrm1-encoding nucleic acid, e.g., through a 2A self-cleaving peptide-encoding sequence. Non-limiting examples of 2A self-cleaving peptides include T2A, P2A, E2A, and F2A. Any self-cleaving peptide sequence known in the art can be used to link Rrm2 to Rrm2. In another embodiment, the construct includes Rrm1 and Rrm2 coding sequences and an internal ribosome entry site. In another embodiment, the construct includes separate promoters (the same or different) driving transcription of the respective Rrm1 and Rrm2 sequences.

SEQ ID NO: 6 is an exemplary nucleic acid sequence comprising a Kozak sequence,
Rrm1, P2A, and Rrm2.
GCTAGCGAATTCGCCACCATGCACGTCATCAAGAGAGACGGGAGGCAGGAAAGAGTCAT
GTTCGATAAAATCACTTCAAGAATCCAGAAACTGTGTTACGGGCTGAACATGGACTTCGT
CGATCCTGCCCAGATTACCATGAAAGTGATCCAGGGACTGTACTCTGGCGTCACCACAGT
GGAGCTGGACACACTGGCCGCTGAAACCGCAGCCACACTGACTACCAAACACCCAGATT
ATGCAATTCTGGCTGCACGGATCGCCGTGAGTAATCTGCATAAGGAGACAAAGAAAGTC
TTCTCAGACGTGATGGAGGACCTGTACAATTATATCAACCCTCACAATGGGAAACATTCA
CCAATGGTCGCTAAGAGCACTCTGGACATTGTGCTGGCCAACAAAGATCGGCTGAACAG
CGCTATCATCTACGACCGGGATTTCAGTTACAACTACTTCGGCTTTAAGACACTGGAGAG
ATCATATCTGCTGAAAATCAATGGGAAGGTGGCCGAACGGCCTCAGCACATGCTGATGA
GAGTCAGCGTGGGCATTCATAAGGAGGACATTGATGCCGCTATCGAAACTTACAACCTG
CTGAGCGAGCGCTGGTTCACCCACGCTTCCCCTACACTGTTTAACGCAGGAACCAATCGA
CCACAGCTGAGCAGCTGCTTCCTGCTGAGCATGAAGGACGATTCCATCGAGGGCATCTAC
GACACCCTGAAACAGTGCGCACTGATTTCTAAGAGTGCCGGCGGGATCGGAGTCGCTGT
GAGTTGTATTCGGGCAACCGGCTCATATATCGCCGGCACAAACGGCAACAGCAACGGGC
TGGTCCCCATGCTGAGGGTGTACAACAATACAGCCCGCTATGTGGATCAGGGAGGCAAC
AAGAGACCAGGAGCATTTGCCATCTACCTGGAACCCTGGCACCTGGACATTTTCGAGTTT
CTGGATCTGAAGAAAAATACTGGCAAAGAGGAACAGAGGGCTCGCGACCTGTTCTTTGC
ACTGTGGATTCCCGACCTGTTCATGAAGAGGGTGGAGACCAACCAGGACTGGAGCCTGA
TGTGCCCCAATGAGTGTCCTGGGCTGGATGAAGTGTGGGGAGAGGAATTTGAAAAACTG
TACGCCAGTTATGAGAAGCAGGGCCGAGTGCGGAAAGTGGTCAAGGCCCAGCAGCTGTG
GTACGCTATCATTGAGAGCCAGACAGAAACTGGCACCCCCTACATGCTGTATAAAGACT
CTTGCAACCGCAAGAGTAACCAGCAGAATCTGGGGACCATCAAATGCAGCAATCTGTGT
ACAGAGATTGTGGAATATACTTCCAAGGATGAGGTCGCCGTGTGTAACCTGGCATCACTG
GCCCTGAATATGTACGTCACAAGCGAGCACACTTATGACTTCAAGAAACTGGCTGAAGT
GACCAAAGTGGTCGTGAGGAATCTGAACAAGATCATTGACATCAACTACTATCCCGTGC
CTGAGGCCTGCCTGAGCAATAAGAGACATAGGCCCATCGGGATTGGAGTGCAGGGCCTG
GCTGACGCATTCATCCTGATGCGCTACCCTTTTGAGTCCGCCGAAGCTCAGCTGCTGAAC
AAGCAGATTTTTGAAACAATCTACTACGGGGCTCTGGAGGCATCTTGTGACCTGGCCAAA
GAACAGGGACCCTACGAGACTTATGAAGGCTCCCCTGTGTCTAAGGGCATCCTGCAGTA
CGATATGTGGAACGTCACACCAACTGACCTGTGGGATTGGAAAGTGCTGAAGGAGAAAA
TTGCAAAGTATGGCATCCGGAACAGCCTGCTGATCGCCCCAATGCCCACTGCCTCTACCG
CTCAGATTCTGGGCAACAATGAGTCCATCGAACCATACACTTCTAACATCTACACCCGGA
GAGTCCTGAGCGGGGAGTTCCAGATCGTGAATCCCCACCTGCTGAAAGACCTGACCGAA
CGGGGACTGTGGCATGAGGAAATGAAGAACCAGATCATTGCCTGCAATGGCAGTATCCA
GTCAATTCCTGAGATCCCAGACGATCTGAAACAGCTGTACAAGACAGTCTGGGAGATCA
GCCAGAAAACTGTGCTGAAGATGGCAGCCGAAAGAGGGGCTTTCATTGATCAGTCACAG
AGCCTGAACATCCACATTGCCGAGCCCAATTACGGAAAGCTGACCTCCATGCATTTTTAT
GGGTGGAAACAGGGACTGAAGACTGGCATGTACTATCTGCGCACCCGACCAGCTGCAAA
CCCCATCCAGTTTACCCTGAATAAGGAGAAACTGAAGGACAAAGAAAAGGTGTCCAAAG
AGGAAGAGGAAAAGGAGAGAAACACAGCCGCTATGGTGTGTTCTCTGGAGAATAGGGA
TGAATGCCTGATGTGTGGCAGTGGAAGCGGAGCTACTAACTTCAGCCTGCTGAAGCAGG
CTGGAGACGTGGAGGAGAACCCTGGACCTCTGAGTCTGAGGGTCCCACTGGCACCTATC
ACCGATCCACAGCAGCTGCAGCTGAGCCCACTGAAAGGCCTGAGTCTGGTCGATAAAGA
GAACACACCACCTGCACTGAGTGGCACTCGGGTGCTGGCATCAAAGACCGCCCGGAGAA
TTTTCCAGGAGCCAACCGAACCCAAAACAAAGGCCGCTGCACCTGGGGTCGAGGACGAA
CCACTGCTGAGAGAGAATCCCAGGCGCTTCGTGATTTTTCCTATCGAATACCACGATATT
TGGCAGATGTATAAGAAAGCTGAGGCAAGTTTCTGGACAGCTGAGGAAGTGGACCTGAG
CAAAGACATCCAGCACTGGGAATCCCTGAAGCCAGAGGAAAGGTACTTCATTTCTCATG
TGCTGGCATTCTTTGCCGCTAGTGACGGGATCGTGAACGAGAATCTGGTCGAACGCTTTA
GCCAGGAGGTGCAGATCACTGAAGCCCGATGCTTCTATGGATTTCAGATTGCTATGGAGA
ACATCCATTCAGAAATGTACAGCCTGCTGATTGACACCTATATCAAAGATCCTAAGGAGC
GCGAGTTCCTGTTTAATGCCATTGAGACAATGCCATGTGTGAAGAAAAAGGCAGACTGG
GCTCTGCGATGGATCGGCGATAAGGAGGCTACTTACGGGGAAAGAGTGGTCGCATTCGC
AGCCGTGGAGGGAATTTTCTTTTCTGGCAGTTTCGCTTCCATCTTTTGGCTGAAAAAGCG
AGGCCTGATGCCTGGGCTGACCTTTTCCAACGAGCTGATTTCTCGCGACGAAGGCCTGCA
CTGCGATTTCGCCTGTCTGATGTTTAAACACCTGGTGCATAAGCCCTCTGAGGAACGAGT
CCGGGAGATCATTATCAACGCAGTGAGGATCGAGCAGGAGTTCCTGACAGAAGCCCTGC
CTGTCAAACTGATTGGCATGAATTGCACTCTGATGAAGCAGTACATCGAGTTTGTGGCCG
ACAGGCTGATGCTGGAACTGGGATTCTCAAAGGTGTTTCGCGTCGAGAACCCATTCGATT
TTATGGAGAATATCAGCCTGGAAGGCAAAACAAACTTCTTTGAGAAGAGAGTCGGGGAA
TATCAGAGGATGGGCGTGATGAGCAGCCCCACTGAGAATAGCTTCACCCTGGACGCCGA
TTTTTGAGCTAGC
SEQ ID NO: 7 is an exemplary Kozak sequence (as found in SEQ ID NO: 6).
GCCACC
SEQ ID NO: 8 is an exemplary RrmQ sequence (as found in SEQ ID NO: 6).
ATGCACGTCATCAAGAGAGACGGGAGGCAGGAAAGAGTCATGTTCGATAAAATCACTTC
AAGAATCCAGAAACTGTGTTACGGGCTGAACATGGACTTCGTCGATCCTGCCCAGATTAC
CATGAAAGTGATCCAGGGACTGTACTCTGGCGTCACCACAGTGGAGCTGGACACACTGG
CCGCTGAAACCGCAGCCACACTGACTACCAAACACCCAGATTATGCAATTCTGGCTGCAC
GGATCGCCGTGAGTAATCTGCATAAGGAGACAAAGAAAGTCTTCTCAGACGTGATGGAG
GACCTGTACAATTATATCAACCCTCACAATGGGAAACATTCACCAATGGTCGCTAAGAGC
ACTCTGGACATTGTGCTGGCCAACAAAGATCGGCTGAACAGCGCTATCATCTACGACCG
GGATTTCAGTTACAACTACTTCGGCTTTAAGACACTGGAGAGATCATATCTGCTGAAAAT
CAATGGGAAGGTGGCCGAACGGCCTCAGCACATGCTGATGAGAGTCAGCGTGGGCATTC
ATAAGGAGGACATTGATGCCGCTATCGAAACTTACAACCTGCTGAGCGAGCGCTGGTTC
ACCCACGCTTCCCCTACACTGTTTAACGCAGGAACCAATCGACCACAGCTGAGCAGCTGC
TTCCTGCTGAGCATGAAGGACGATTCCATCGAGGGCATCTACGACACCCTGAAACAGTG
CGCACTGATTTCTAAGAGTGCCGGCGGGATCGGAGTCGCTGTGAGTTGTATTCGGGCAAC
CGGCTCATATATCGCCGGCACAAACGGCAACAGCAACGGGCTGGTCCCCATGCTGAGGG
TGTACAACAATACAGCCCGCTATGTGGATCAGGGAGGCAACAAGAGACCAGGAGCATTT
GCCATCTACCTGGAACCCTGGCACCTGGACATTTTCGAGTTTCTGGATCTGAAGAAAAAT
ACTGGCAAAGAGGAACAGAGGGCTCGCGACCTGTTCTTTGCACTGTGGATTCCCGACCTG
TTCATGAAGAGGGTGGAGACCAACCAGGACTGGAGCCTGATGTGCCCCAATGAGTGTCC
TGGGCTGGATGAAGTGTGGGGAGAGGAATTTGAAAAACTGTACGCCAGTTATGAGAAGC
AGGGCCGAGTGCGGAAAGTGGTCAAGGCCCAGCAGCTGTGGTACGCTATCATTGAGAGC
CAGACAGAAACTGGCACCCCCTACATGCTGTATAAAGACTCTTGCAACCGCAAGAGTAA
CCAGCAGAATCTGGGGACCATCAAATGCAGCAATCTGTGTACAGAGATTGTGGAATATA
CTTCCAAGGATGAGGTCGCCGTGTGTAACCTGGCATCACTGGCCCTGAATATGTACGTCA
CAAGCGAGCACACTTATGACTTCAAGAAACTGGCTGAAGTGACCAAAGTGGTCGTGAGG
AATCTGAACAAGATCATTGACATCAACTACTATCCCGTGCCTGAGGCCTGCCTGAGCAAT
AAGAGACATAGGCCCATCGGGATTGGAGTGCAGGGCCTGGCTGACGCATTCATCCTGAT
GCGCTACCCTTTTGAGTCCGCCGAAGCTCAGCTGCTGAACAAGCAGATTTTTGAAACAAT
CTACTACGGGGCTCTGGAGGCATCTTGTGACCTGGCCAAAGAACAGGGACCCTACGAGA
CTTATGAAGGCTCCCCTGTGTCTAAGGGCATCCTGCAGTACGATATGTGGAACGTCACAC
CAACTGACCTGTGGGATTGGAAAGTGCTGAAGGAGAAAATTGCAAAGTATGGCATCCGG
AACAGCCTGCTGATCGCCCCAATGCCCACTGCCTCTACCGCTCAGATTCTGGGCAACAAT
GAGTCCATCGAACCATACACTTCTAACATCTACACCCGGAGAGTCCTGAGCGGGGAGTTC
CAGATCGTGAATCCCCACCTGCTGAAAGACCTGACCGAACGGGGACTGTGGCATGAGGA
AATGAAGAACCAGATCATTGCCTGCAATGGCAGTATCCAGTCAATTCCTGAGATCCCAG
ACGATCTGAAACAGCTGTACAAGACAGTCTGGGAGATCAGCCAGAAAACTGTGCTGAAG
ATGGCAGCCGAAAGAGGGGCTTTCATTGATCAGTCACAGAGCCTGAACATCCACATTGC
CGAGCCCAATTACGGAAAGCTGACCTCCATGCATTTTTATGGGTGGAAACAGGGACTGA
AGACTGGCATGTACTATCTGCGCACCCGACCAGCTGCAAACCCCATCCAGTTTACCCTGA
ATAAGGAGAAACTGAAGGACAAAGAAAAGGTGTCCAAAGAGGAAGAGGAAAAGGAGA
GAAACACAGCCGCTATGGTGTGTTCTCTGGAGAATAGGGATGAATGCCTGATGTGTGGC
AGT
SEQ ID NO: 9 is an exemplary P2A sequence (as found in SEQ ID NO: 6).
GCTACTAACTTCAGCCTGCTGAAGCAGGCTGGAGACGTGGAGGAGAACCCTGGACCT

### Rrm2 Mutants

Mutations found within the ubiquitin binding domain (i.e., the site of ubiquitin addition or ubiquitination) of Rrm2 decrease ubiquitination of Rrm2, increase Rrm2 stability (e.g., half-life of Rrm2), and result in increased dATP in the cell. Accordingly, provided herein is an isolated nucleic acid molecule encoding an Rrm2 polypeptide that, together with Rrm1 polypeptide comprises ribonucleotide reductase activity. In some embodiments, the encoded Rrm2 polypeptide comprises a mutation that increases the intracellular level of the polypeptide as compared to wild-type Rrm2 polypeptide. In one embodiment, the mutation is in a ubiquitin binding degron of Rrm2. In one embodiment, the ubiquitin binding degrons of Rrm2 are found at nucleotides 88-96 (which encode amino acids that can associate with the APC/FZR1 proteasome) and nucleotides 97-99 and 145-153 (which can associate with the SCF/CyclinF proteasome) of wild-type Rrm2 (SEQ ID NOs: 3). In one embodiment, the ubiquitin binding degrons of Rrm2 are found at amino acids 30-32 (which can associate with the APC/FZR1 proteasome) and amino acids 33 and 49-51 (which can associate with the SCF/CyclinF proteasome) of wild-type Rrm2 (SEQ ID NO: 4).

Where a primary function of the RNR complex is to provide deoxyribonucleotides for DNA synthesis, the degradation of RRM2 polypeptide subunit is tightly regulated in a cell-cycle dependent manner. Without wishing to be bound by theory, that regulation is believed to occur at least in part via the activity of the Cdh1-associated anaphase-promoting complex (Cdh1-APC) on a KEN-box APC recognition signal in the RRM2 polypeptide. See, e.g., Chabes et al., Proc. Natl. Acad Sci. U.S.A. 100: 3925-3929 (2003), and Pfleger, CM & Kirschner, MW, Genes Dev. 14: 655-665 (2000), each of which is incorporated herein by reference.

In one embodiment, the ubiquitin binding degrons of Rrm2 have the nucleic acid sequence of SEQ ID NOs: 10-12 or the amino acid sequence of SEQ ID NOs: 13-15:

SEQ ID NO: 10
AAGGAGAAC;
SEQ ID NO: 11
ACG;
SEQ ID NO: 12
AGGAGGATC;
SEQ ID NO: 13
KEN;
SEQ ID NO: 14
T;
SEQ ID NO: 15
RRI.

In one embodiment, the mutant Rrm2 having mutations within the ubiquitin binding degrons of Rrm2 have one mutation, at least one mutation, at least 2 mutations, at least 3 mutations, at least 4 mutations, at least 5 mutations, at least 6 mutations, at least 7 mutations, at least 8 mutations, at least 9 mutations, or 10 mutations, or more. Alternatively, the mutant Rrm2 can include 1 mutation, two or fewer mutations, three or fewer mutations, four or fewer mutations, five or fewer mutations, six or fewer mutations, seven or fewer mutations, eight or fewer mutations, nine or fewer mutations or 10 or fewer mutations, relative to wild-type Rrm2 polypeptide. Mutations found within a ubiquitin binding degron can be in succession, e.g., two or three amino acids in a row can be mutated. Alternatively, mutations found within a ubiquitin binding degron can have wild-type amino acids in between. A mutation described herein can be an amino acid substitution, deletion, or insertion. It is contemplated herein that a mutation can be any amino acid change within the ubiquitin binding domain that results in at least decreased ubiquitination of Rrm2, increased stability of Rrm2, and/or increased dATP levels in the cell. Considerations for mutating a ubiquitination site while maintaining Rrm2 activity in terms of complex formation and ribonucleotide reductase activity with Rim1 are discussed herein above. In some embodiments, the mutation is found near a ubiquitin binding degron, e.g., within 1-10 nucleotides of a ubiquitin binding degron, i.e., nucleotides not encoding a ubiquitin binding degron. In some embodiments, the mutation is found near a ubiquitin binding degron, e.g., within 1-10 amino acids of a ubiquitin binding degron, i.e., amino acids not encoding a ubiquitin binding degron.

In one embodiment, the mutation is selected from Table 1.

TABLE 1
Rrm2 mutations (amino acid and its corresponding
number in SEQ ID NO: 3 shown)

| Positon | 30 | 31 | 32 | 33 | 49 | 51 |
|---|---|---|---|---|---|---|
| Wild-type | K | E | N | T | R | I |
| Mutant | A | A | A | A | A | A |

In one embodiment, the isolated nucleic acid has a sequence of SEQ ID NOs: 16-20. In one embodiment, the isolated nucleic acid consists of or consists essentially of the sequence of SEQ ID NO: 16-20.

SEQ ID NO: 16 is an exemplary nucleic acid sequence for Rrm2 mutant 30AAA -
ATGCTGAGTCTGAGGGTCCCACTGGCACCTATCACCGATCCACAGCAGCTGCAGCTGAGC
CCACTGAAAGGCCTGAGTCTGGTCGATGCAGCGGCCACACCACCTGCACTGAGTGGCAC
TCGGGTGCTGGCATCAAAGACCGCCCGGAGAATTTTCCAGGAGCCAACCGAACCCAAAA
CAAAGGCCGCTGCACCTGGGGTCGAGGACGAACCACTGCTGAGAGAGAATCCCAGGCGC
TTCGTGATTTTTCCTATCGAATACCACGATATTTGGCAGATGTATAAGAAAGCTGAGGCA
AGTTTCTGGACAGCTGAGGAAGTGGACCTGAGCAAAGACATCCAGCACTGGGAATCCCT
GAAGCCAGAGGAAAGGTACTTCATTTCTCATGTGCTGGCATTCTTTGCCGCTAGTGACGG
GATCGTGAACGAGAATCTGGTCGAACGCTTTAGCCAGGAGGTGCAGATCACTGAAGCCC
GATGCTTCTATGGATTTCAGATTGCTATGGAGAACATCCATTCAGAAATGTACAGCCTGC
TGATTGACACCTATATCAAAGATCCTAAGGAGCGCGAGTTCCTGTTTAATGCCATTGAGA
CAATGCCATGTGTGAAGAAAAAGGCAGACTGGGCTCTGCGATGGATCGGCGATAAGGAG
GCTACTTACGGGGAAAGAGTGGTCGCATTCGCAGCCGTGGAGGGAATTTTCTTTTCTGGC
AGTTTCGCTTCCATCTTTTGGCTGAAAAAGCGAGGCCTGATGCCTGGGCTGACCTTTTCC
AACGAGCTGATTTCTCGCGACGAAGGCCTGCACTGCGATTTCGCCTGTCTGATGTTTAAA
CACCTGGTGCATAAGCCCTCTGAGGAACGAGTCCGGGAGATCATTATCAACGCAGTGAG
GATCGAGCAGGAGTTCCTGACAGAAGCCCTGCCTGTCAAACTGATTGGCATGAATTGCA
CTCTGATGAAGCAGTACATCGAGTTTGTGGCCGACAGGCTGATGCTGGAACTGGGATTCT
CAAAGGTGTTTCGCGTCGAGAACCCATTCGATTTTATGGAGAATATCAGCCTGGAAGGCA
AAACAAACTTCTTTGAGAAGAGAGTCGGGGAATATCAGAGGATGGGCGTGATGAGCAGC
CCCACTGAGAATAGCTTCACCCTGGACGCCGATTTTTGA
SEQ ID NO: 17 is an exemplary nucleic acid sequence for Rrm2 mutant 30AAN-
ATGCTGAGTCTGAGGGTCCCACTGGCACCTATCACCGATCCACAGCAGCTGCAGCTGAGC
CCACTGAAAGGCCTGAGTCTGGTCGATGCAGCGAACACACCACCTGCACTGAGTGGCAC
TCGGGTGCTGGCATCAAAGACCGCCCGGAGAATTTTCCAGGAGCCAACCGAACCCAAAA
CAAAGGCCGCTGCACCTGGGGTCGAGGACGAACCACTGCTGAGAGAGAATCCCAGGCGC
TTCGTGATTTTTCCTATCGAATACCACGATATTTGGCAGATGTATAAGAAAGCTGAGGCA
AGTTTCTGGACAGCTGAGGAAGTGGACCTGAGCAAAGACATCCAGCACTGGGAATCCCT
GAAGCCAGAGGAAAGGTACTTCATTTCTCATGTGCTGGCATTCTTTGCCGCTAGTGACGG
GATCGTGAACGAGAATCTGGTCGAACGCTTTAGCCAGGAGGTGCAGATCACTGAAGCCC
GATGCTTCTATGGATTTCAGATTGCTATGGAGAACATCCATTCAGAAATGTACAGCCTGC
TGATTGACACCTATATCAAAGATCCTAAGGAGCGCGAGTTCCTGTTTAATGCCATTGAGA
CAATGCCATGTGTGAAGAAAAAGGCAGACTGGGCTCTGCGATGGATCGGCGATAAGGAG
GCTACTTACGGGGAAAGAGTGGTCGCATTCGCAGCCGTGGAGGGAATTTTCTTTTCTGGC
AGTTTCGCTTCCATCTTTTGGCTGAAAAAGCGAGGCCTGATGCCTGGGCTGACCTTTTCC
AACGAGCTGATTTCTCGCGACGAAGGCCTGCACTGCGATTTCGCCTGTCTGATGTTTAAA
CACCTGGTGCATAAGCCCTCTGAGGAACGAGTCCGGGAGATCATTATCAACGCAGTGAG
GATCGAGCAGGAGTTCCTGACAGAAGCCCTGCCTGTCAAACTGATTGGCATGAATTGCA
CTCTGATGAAGCAGTACATCGAGTTTGTGGCCGACAGGCTGATGCTGGAACTGGGATTCT
CAAAGGTGTTTCGCGTCGAGAACCCATTCGATTTTATGGAGAATATCAGCCTGGAAGGCA
AAACAAACTTCTTTGAGAAGAGAGTCGGGGAATATCAGAGGATGGGCGTGATGAGCAGC
CCCACTGAGAATAGCTTCACCCTGGACGCCGATTTTTGA
SEQ ID NO: 18 is an exemplary nucleic acid sequence for Rrm2 mutant 33A -
ATGCTGAGTCTGAGGGTCCCACTGGCACCTATCACCGATCCACAGCAGCTGCAGCTGAGC
CCACTGAAAGGCCTGAGTCTGGTCGATAAAGAGAACGCACCACCTGCACTGAGTGGCAC
TCGGGTGCTGGCATCAAAGACCGCCCGGAGAATTTTCCAGGAGCCAACCGAACCCAAAA
CAAAGGCCGCTGCACCTGGGGTCGAGGACGAACCACTGCTGAGAGAGAATCCCAGGCGC
TTCGTGATTTTTCCTATCGAATACCACGATATTTGGCAGATGTATAAGAAAGCTGAGGCA
AGTTTCTGGACAGCTGAGGAAGTGGACCTGAGCAAAGACATCCAGCACTGGGAATCCCT
GAAGCCAGAGGAAAGGTACTTCATTTCTCATGTGCTGGCATTCTTTGCCGCTAGTGACGG
GATCGTGAACGAGAATCTGGTCGAACGCTTTAGCCAGGAGGTGCAGATCACTGAAGCCC
GATGCTTCTATGGATTTCAGATTGCTATGGAGAACATCCATTCAGAAATGTACAGCCTGC
TGATTGACACCTATATCAAAGATCCTAAGGAGCGCGAGTTCCTGTTTAATGCCATTGAGA
CAATGCCATGTGTGAAGAAAAAGGCAGACTGGGCTCTGCGATGGATCGGCGATAAGGAG
GCTACTTACGGGGAAAGAGTGGTCGCATTCGCAGCCGTGGAGGGAATTTTCTTTTCTGGC
AGTTTCGCTTCCATCTTTTGGCTGAAAAAGCGAGGCCTGATGCCTGGGCTGACCTTTTCC
AACGAGCTGATTTCTCGCGACGAAGGCCTGCACTGCGATTTCGCCTGTCTGATGTTTAAA
CACCTGGTGCATAAGCCCTCTGAGGAACGAGTCCGGGAGATCATTATCAACGCAGTGAG
GATCGAGCAGGAGTTCCTGACAGAAGCCCTGCCTGTCAAACTGATTGGCATGAATTGCA
CTCTGATGAAGCAGTACATCGAGTTTGTGGCCGACAGGCTGATGCTGGAACTGGGATTCT
CAAAGGTGTTTCGCGTCGAGAACCCATTCGATTTTATGGAGAATATCAGCCTGGAAGGCA
AAACAAACTTCTTTGAGAAGAGAGTCGGGGAATATCAGAGGATGGGCGTGATGAGCAGC
CCCACTGAGAATAGCTTCACCCTGGACGCCGATTTTTGA
SEQ ID NO: 19 is an exemplary nucleic acid sequence for Rrm2 mutant 49ARA -
ATGCTGAGTCTGAGGGTCCCACTGGCACCTATCACCGATCCACAGCAGCTGCAGCTGAGC
CCACTGAAAGGCCTGAGTCTGGTCGATAAAGAGAACACACCACCTGCACTGAGTGGCAC
TCGGGTGCTGGCATCAAAGACCGCCGCGAGAGCTTTCCAGGAGCCAACCGAACCCAAAA
CAAAGGCCGCTGCACCTGGGGTCGAGGACGAACCACTGCTGAGAGAGAATCCCAGGCGC
TTCGTGATTTTTCCTATCGAATACCACGATATTTGGCAGATGTATAAGAAAGCTGAGGCA
AGTTTCTGGACAGCTGAGGAAGTGGACCTGAGCAAAGACATCCAGCACTGGGAATCCCT
GAAGCCAGAGGAAAGGTACTTCATTTCTCATGTGCTGGCATTCTTTGCCGCTAGTGACGG
GATCGTGAACGAGAATCTGGTCGAACGCTTTAGCCAGGAGGTGCAGATCACTGAAGCCC
GATGCTTCTATGGATTTCAGATTGCTATGGAGAACATCCATTCAGAAATGTACAGCCTGC
TGATTGACACCTATATCAAAGATCCTAAGGAGCGCGAGTTCCTGTTTAATGCCATTGAGA
CAATGCCATGTGTGAAGAAAAAGGCAGACTGGGCTCTGCGATGGATCGGCGATAAGGAG
GCTACTTACGGGGAAAGAGTGGTCGCATTCGCAGCCGTGGAGGGAATTTTCTTTTCTGGC
AGTTTCGCTTCCATCTTTTGGCTGAAAAAGCGAGGCCTGATGCCTGGGCTGACCTTTTCC
AACGAGCTGATTTCTCGCGACGAAGGCCTGCACTGCGATTTCGCCTGTCTGATGTTTAAA
CACCTGGTGCATAAGCCCTCTGAGGAACGAGTCCGGGAGATCATTATCAACGCAGTGAG
GATCGAGCAGGAGTTCCTGACAGAAGCCCTGCCTGTCAAACTGATTGGCATGAATTGCA
CTCTGATGAAGCAGTACATCGAGTTTGTGGCCGACAGGCTGATGCTGGAACTGGGATTCT
CAAAGGTGTTTCGCGTCGAGAACCCATTCGATTTTATGGAGAATATCAGCCTGGAAGGCA
AAACAAACTTCTTTGAGAAGAGAGTCGGGGAATATCAGAGGATGGGCGTGATGAGCAGC
CCCACTGAGAATAGCTTCACCCTGGACGCCGATTTTTGA
SEQ ID NO: 20 is an exemplary nucleic acid sequence for Rrm2 mutant
30AAA/49ARA-
ATGCTGAGTCTGAGGGTCCCACTGGCACCTATCACCGATCCACAGCAGCTGCAGCTGAGC
CCACTGAAAGGCCTGAGTCTGGTCGATGCAGCGGCCACACCACCTGCACTGAGTGGCAC
TCGGGTGCTGGCATCAAAGACCGCCGCGAGAGCTTTCCAGGAGCCAACCGAACCCAAAA
CAAAGGCCGCTGCACCTGGGGTCGAGGACGAACCACTGCTGAGAGAGAATCCCAGGCGC
TTCGTGATTTTTCCTATCGAATACCACGATATTTGGCAGATGTATAAGAAAGCTGAGGCA
AGTTTCTGGACAGCTGAGGAAGTGGACCTGAGCAAAGACATCCAGCACTGGGAATCCCT
GAAGCCAGAGGAAAGGTACTTCATTTCTCATGTGCTGGCATTCTTTGCCGCTAGTGACGG
GATCGTGAACGAGAATCTGGTCGAACGCTTTAGCCAGGAGGTGCAGATCACTGAAGCCC
GATGCTTCTATGGATTTCAGATTGCTATGGAGAACATCCATTCAGAAATGTACAGCCTGC
TGATTGACACCTATATCAAAGATCCTAAGGAGCGCGAGTTCCTGTTTAATGCCATTGAGA
CAATGCCATGTGTGAAGAAAAAGGCAGACTGGGCTCTGCGATGGATCGGCGATAAGGAG
GCTACTTACGGGGAAAGAGTGGTCGCATTCGCAGCCGTGGAGGGAATTTTCTTTTCTGGC
AGTTTCGCTTCCATCTTTTGGCTGAAAAAGCGAGGCCTGATGCCTGGGCTGACCTTTTCC
AACGAGCTGATTTCTCGCGACGAAGGCCTGCACTGCGATTTCGCCTGTCTGATGTTTAAA
CACCTGGTGCATAAGCCCTCTGAGGAACGAGTCCGGGAGATCATTATCAACGCAGTGAG
GATCGAGCAGGAGTTCCTGACAGAAGCCCTGCCTGTCAAACTGATTGGCATGAATTGCA
CTCTGATGAAGCAGTACATCGAGTTTGTGGCCGACAGGCTGATGCTGGAACTGGGATTCT
CAAAGGTGTTTCGCGTCGAGAACCCATTCGATTTTATGGAGAATATCAGCCTGGAAGGCA
AAACAAACTTCTTTGAGAAGAGAGTCGGGGAATATCAGAGGATGGGCGTGATGAGCAGC
CCCACTGAGAATAGCTTCACCCTGGACGCCGATTTTTGA

In one embodiment, an isolated nucleic acid comprises, consists of, or consists essentially of a sequence at least 90%, at least 91%, at least 92%, at least 93%, at least 94%, at least 95%, at least 96%, at least 97%, at least 98%, at least 99%, or more, identical to one of SEQ ID NO: 16-20, and retains at least 50% of the function of one of SEQ ID NO: 16-20, e.g., complex formation with Rrm1 and increasing the cellular level of dATP.

In one embodiment, the isolated polypeptide has a sequence of SEQ ID NOs: 21-25. In one embodiment, the isolated polypeptide consists of or consists essentially of a polypeptide having the sequence of SEQ ID NO: 21-25.

SEQ ID NO: 21 is an exemplary amino acid sequence for Rrm2 mutant 30AAA-
MLSLRVPLAPITDPQQLQLSPLKGLSLVDAAATPPALSGTRVLASKTARRIFQEPTEPKTKAA
APGVEDEPLLRENPRRFVIFPIEYHDIWQMYKKAEASFWTAEEVDLSKDIQHWESLKPEERYF
ISHVLAFFAASDGIVNENLVERFSQEVQITEARCFYGFQIAMENIHSEMYSLLIDTYIKDPKERE
FLFNAIETMPCVKKKADWALRWIGDKEATYGERVVAFAAVEGIFFSGSFASIFWLKKRGLMP
GLTFSNELISRDEGLHCDFACLMFKHLVHKPSEERVREIIINAVRIEQEFLTEALPVKLIGMNCT
LMKQYIEFVADRLMLELGFSKVFRVENPFDFMENISLEGKTNFFEKRVGEYQRMGVMSSPTE
NSFTLDADF
SEQ ID NO: 22 is an exemplary amino acid sequence for Rrm2 mutant 30AAN-
MLSLRVPLAPITDPQQLQLSPLKGLSLVDAANTPPALSGTRVLASKTARRIFQEPTEPKTKAA
APGVEDEPLLRENPRRFVIFPIEYHDIWQMYKKAEASFWTAEEVDLSKDIQHWESLKPEERYF
ISHVLAFFAASDGIVNENLVERFSQEVQITEARCFYGFQIAMENIHSEMYSLLIDTYIKDPKERE
FLFNAIETMPCVKKKADWALRWIGDKEATYGERVVAFAAVEGIFFSGSFASIFWLKKRGLMP
GLTFSNELISRDEGLHCDFACLMFKHLVHKPSEERVREIIINAVRIEQEFLTEALPVKLIGMNCT
LMKQYIEFVADRLMLELGFSKVFRVENPFDFMENISLEGKTNFFEKRVGEYQRMGVMSSPTE
NSFTLDADF
SEQ ID NO: 23 is an exemplary amino acid sequence for Rrm2 mutant 33A-
MLSLRVPLAPITDPQQLQLSPLKGLSLVDKENAPPALSGTRVLASKTARRIFQEPTEPKTKAA
APGVEDEPLLRENPRRFVIFPIEYHDIWQMYKKAEASFWTAEEVDLSKDIQHWESLKPEERYF
ISHVLAFFAASDGIVNENLVERFSQEVQITEARCFYGFQIAMENIHSEMYSLLIDTYIKDPKERE
FLFNAIETMPCVKKKADWALRWIGDKEATYGERVVAFAAVEGIFFSGSFASIFWLKKRGLMP
GLTFSNELISRDEGLHCDFACLMFKHLVHKPSEERVREIIINAVRIEQEFLTEALPVKLIGMNCT
LMKQYIEFVADRLMLELGFSKVFRVENPFDFMENISLEGKTNFFEKRVGEYQRMGVMSSPTE
NSFTLDADF
SEQ ID NO: 24 is an exemplary amino acid sequence for Rrm2 mutant 49ARA-
MLSLRVPLAPITDPQQLQLSPLKGLSLVDKENTPPALSGTRVLASKTAARAFQEPTEPKTKAA
APGVEDEPLLRENPRRFVIFPIEYHDIWQMYKKAEASFWTAEEVDLSKDIQHWESLKPEERYF
ISHVLAFFAASDGIVNENLVERFSQEVQITEARCFYGFQIAMENIHSEMYSLLIDTYIKDPKERE
FLFNAIETMPCVKKKADWALRWIGDKEATYGERVVAFAAVEGIFFSGSFASIFWLKKRGLMP
GLTFSNELISRDEGLHCDFACLMFKHLVHKPSEERVREIIINAVRIEQEFLTEALPVKLIGMNCT
LMKQYIEFVADRLMLELGFSKVFRVENPFDFMENISLEGKTNFFEKRVGEYQRMGVMSSPTE
NSFTLDADF
SEQ ID NO: 25 is an exemplary amino acid sequence for Rrm2 mutant
30AAA/49ARA-
MLSLRVPLAPITDPQQLQLSPLKGLSLVDAAATPPALSGTRVLASKTAARAFQEPTEPKTKAA
APGVEDEPLLRENPRRFVIFPIEYHDIWQMYKKAEASFWTAEEVDLSKDIQHWESLKPEERYF
ISHVLAFFAASDGIVNENLVERFSQEVQITEARCFYGFQIAMENIHSEMYSLLIDTYIKDPKERE
FLFNAIETMPCVKKKADWALRWIGDKEATYGERVVAFAAVEGIFFSGSFASIFWLKKRGLMP
GLTFSNELISRDEGLHCDFACLMFKHLVHKPSEERVREIIINAVRIEQEFLTEALPVKLIGMNCT
LMKQYIEFVADRLMLELGFSKVFRVENPFDFMENISLEGKTNFFEKRVGEYQRMGVMSSPTE
NSFTLDADF

In one embodiment, an isolated polypeptide comprises, consists of, or consists essentially of polypeptide having a sequence having at least 90%, at least 91%, at least 92%, at least 93%, at least 94%, at least 95%, at least 96%, at least 97%, at least 98%, at least 99%, or more, identical to one of SEQ ID NO: 21-25, and retains as least 50% of the function of one of SEQ ID NO: 21-25, e.g., complex formation with Rrm1 and increasing the cellular level of dATP.

SEQ ID NO: 26 is an exemplary nucleotide sequence
for Rrm2 mutant 30AAA/49ARA:
(SEQ ID NO: 26)
ctgagtctgagggtcccactggcacctatcaccgatccacagcagctgca
gctgagcccactgaaaggcctgagtctggtcgatgcagcggccacaccac
ctgcactgagtggcactcgggtgctggcatcaaagaccgccgcgagagct
ttccaggagccaaccgaacccaaaacaaaggccgctgcacctggggtcga
ggacgaaccactgctgagagagaatcccaggcgcttcgtgatttttccta
tcgaataccacgatatttggcagatgtataagaaagctgaggcaagtttc
tggacagctgaggaagtggacctgagcaaagacatccagcactgggaatc
cctgaagccagaggaaaggtacttcatttctcatgtgctggcattctttg
ccgctagtgacgggatcgtgaacgagaatctggtcgaacgctttagccag
gaggtgcagatcactgaagcccgatgcttctatggatttcagattgctat
ggagaacatccattcagaaatgtacagcctgctgattgacacctatatca
aagatcctaaggagcgcgagttcctgtttaatgccattgagacaatgcca
tgtgtgaagaaaaaggcagactgggctctgcgatggatcggcgataagga
ggctacttacggggaaagagtggtcgcattcgcagccgtggagggaattt
tcttttctggcagtttcgcttccatcttttggctgaaaaagcgaggcctg
atgcctgggctgaccttttccaacgagctgatttctcgcgacgaaggcct
gcactgcgatttcgcctgtctgatgtttaaacacctggtgcataagccct
ctgaggaacgagtccgggagatcattatcaacgcagtgaggatcgagcag
gagttcctgacagaagccctgcctgtcaaactgattggcatgaattgcac
tctgatgaagcagtacatcgagtttgtggccgacaggctgatgctggaac
tgggattctcaaaggtgtttcgcgtcgagaacccattcgattttatggag
aatatcagcctggaaggcaaaacaaacttctttgagaagagagtcgggga
atatcagaggatgggcgtgatgagcagccccactgagaatagcttcaccc
tggacgccgatttttg

### Muscle-Specific Promoters

A regulatory cassette directs the expression of a transgene (e.g., RRM1, RRM2). A regulatory cassette generally comprises a promoter element and other sequences necessary to direct the assembly of an active transcriptase complex in a desired cell type. A regulatory cassette can also include, for example, a 3′ untranslated sequence including a polyadenylation signal downstream of the region where an open reading frame encoding the desired polypeptide is or can be inserted.

A “muscle-specific” promoter or regulatory cassette or element is one that is at least 10× more active in a muscle cell than in a non-muscle cell, e.g., at least 20×, 30×, 40×, 50×, 60×, 70×, 80×, 90×, 100×, 200×, 300×, 400×, 500×, 600×, 700×, 800×, 900× or 1000× greater activity in a muscle cell relative to a non-muscle cell. In some embodiments, a muscle-specific promoter or regulatory cassette or element is not substantially active in non-muscle cells. By “not substantially active” in the context of promoter or regulatory cassette activity is meant that transcription products are not detectable, e.g., by RT-PCR. “Cardiac muscle-specific” promoters or regulatory cassettes or elements are a subset of muscle-specific promoters, cassettes or elements. Some cardiac-specific promoter or regulatory cassettes are active to some extent in non-cardiac muscle cells, but are active to a greater extent in cardiac muscle cells, and others are substantially active only in cardiac muscle cells, i.e., are not substantially active in other muscle cell types. Thus, a “cardiac muscle-specific” promoter or regulatory cassette is at least 2× more active in a cardiac muscle cell than in a muscle cells that is not a cardiac muscle cell, e.g., at least 3×, 4×, 5×, 6×, 7×, 8×, 9×, 10×, 20×, 30×, 40×, 50×, 60×, 70×, 80×, 90× or 100× more active in a cardiac muscle cell than in a muscle cell that is not a cardiac muscle cell, and a cardiac muscle specific promoter or regulatory cassette can, in some embodiments, only be active in a cardiac muscle cell.

Muscle-specific expression cassettes (MSECs) are engineered transcriptional regulatory elements that drive expression in a muscle-specific manner. Various MSECs have varying specificity for different types of muscle cells or tissue, such as skeletal and cardiac muscle tissue. In some embodiments, an MSEC as described herein can comprise control elements (e.g., MSEC enhancers and promoters) that bind both ubiquitous and/or muscle type-specific transcription factors; the activity of each MSEC is determined by differences in control element types, sequences, numbers, and linear order within the enhancer and promoter regions. MSECs can be used to avoid toxicity and immune activation that occurs with uncontrolled expression of muscle therapeutic proteins in other sites, as well as to restrict the expression of a desired gene (e.g., a ribonucleotide reductase subunit or subunits) to muscle (e.g., skeletal and/or cardiac). MSECs, their makeup, and how their activity is measured are described in, e.g., PCT/US2019/023915, which is incorporated herein by reference in its entirety.

MSECs useful in the methods and compositions described herein can be modeled on naturally occurring skeletal and/or cardiac muscle gene enhancers and promoters, or can be designed using synthetic arrays of control elements. MSECs can be attached to any cDNA and when the constructs are transduced into cardiac or skeletal muscle cells, the cDNA-encoded protein product will be synthesized. There are currently more than 350 MSECs known, and some can produce product levels over concentration ranges exceeding 1000-fold.

In some embodiments, the muscle-specific transcriptional regulatory cassette is derived from an M-creatine kinase enhancer and/or a M-creatine kinase promoter sequence. For example, the muscle-specific transcriptional regulatory cassette can be derived from an M-creatine kinase enhancer with an M-creatine kinase promoter. Furthermore, the muscle-specific transcriptional regulatory cassette can include one or more enhancers derived from conserved regions of muscle creatine kinase and/or a CK8 transcriptional regulatory cassette.

The muscle-specific transcriptional regulatory cassette can be a muscle-specific CK8 transcriptional regulatory cassette (CK8) or a derivative thereof. CK8 is a non-naturally occurring nucleotide sequence including multiple muscle and non-muscle gene control elements arranged in a miniaturized array. CK8 can provide high or very high transcriptional expression of a predetermined RNA and/or protein in skeletal and cardiac muscle cells. In one embodiment, an MSEC useful for the methods and compositions described herein comprises a modified CK8 transcriptional regulatory cassette (e.g., CK8m and CK8e).

Examples of MSECs include, but are not limited to the following: MSEC-3363, MSEC-74, MSEC-87, MSEC-95, MSEC-118, MSEC-144, MSEC-206A, MSEC-206B, MSEC-212, MSEC-220, MSEC-239, MSEC-259, MSEC-283, MSEC-285, MSEC-288, MSEC-290, MSEC-297A, MSEC-297B, MSEC 297C, MSEC 297D, MSEC 297E, MSEC 297F, MSEC 297G, MSEC 297H, MSEC 2971, MSEC 297J, MSEC 297K, MSEC 297L, MSEC-302A, MSEC-302B, MSEC-302C, MSEC-303, MSEC-304, MSEC-310A, MSEC-31 OB, MSEC 310C, MSEC 310D, MSEC 310E, MSEC 310F, MSEC 310G, MSEC 310H, MSEC 3101, MSEC 310J, MSEC 310K, MSEC 310L, MSEC-315A, MSEC-315B, MSEC-315C, MSEC-318, MSEC-319B, MSEC-320, MSEC-322, MSEC-327, MSEC-330, MSEC-335A, MSEC-335B, MSEC-336A, MSEC-336B, MSEC-336C, MSEC-336D, MSEC-339, MSEC-340, MSEC-341, MSEC-344, MSEC-345, MSEC-346A, MSEC-347, MSEC-348, MSEC-350, MSEC-351, MSEC-352A, MSEC-352B, MSEC-356, MSEC-360A, MSEC-360B, MSEC-361, MSEC-362A, MSEC-362B, MSEC-362C, MSEC-362D, MSEC-362E, MSEC-362F, MSEC-365A, MSEC-365B, MSEC-367, MSEC-378, MSEC-382, MSEC-383, MSEC-384, MSEC-386, MSEC-388, MSEC-393A, MSEC-393B, MSEC-395, MSEC-395B, MSEC-400, MSEC-402, MSEC-403, MSEC-403B, MSEC-405A, MSEC-405B, MSEC-405C, MSEC-406, MSEC-410, MSEC-411, MSEC-413, MSEC-417, MSEC-420A, MSEC-420B, MSEC-421 A, MSEC-421 B, MSEC-421C, MSEC-421 D, MSEC-423, MSEC-425, MSEC-427A, MSEC-427B, MSEC-427C, MSEC-429A, MSEC-429B, MSEC-433, MSEC-438A, MSEC-438B, MSEC-438C, MSEC-438D, MSEC-438E, MSEC-438F, MSEC-438G, MSEC-438H, MSEC-4381, MSEC-439A, MSEC-439B, MSEC-440, MSEC-443, MSEC-444, MSEC-446, MSEC-449, MSEC-450A, MSEC-450B, MSEC-450C, MSEC-450D, MSEC-455A, MSEC-455B, MSEC-455C, MSEC-457, MSEC-461, MSEC-462A, MSEC-462B, MSEC-463, MSEC-464A, MSEC-464 B, MSEC-466, MSEC-472, MSEC-474, MSEC-476, MSEC-476B, MSEC-478A, MSEC-478B, MSEC-478C, MSEC-480, MSEC-481, MSEC-486A, MSEC-486B, MSEC-486C, MSEC-487A, MSEC-487B, MSEC-493A, MSEC-493B, MSEC-495, MSEC-501, MSEC-503, MSEC-508, MSEC-510, MSEC-513, MSEC-517, MSEC-518, MSEC-523, MSEC-527A, MSEC-527B, MSEC-529A, MSEC-529B, MSEC-529C, MSEC-531A, MSEC-531 B, MSEC-538A, MSEC-538B, MSEC-539A, MSEC-539B, MSEC-540, MSEC-541 A, MSEC-541 B, MSEC-546, MSEC-547, MSEC-550, MSEC-553, MSEC-555, MSEC-556A, MSEC-556B, MSEC-556C, MSEC-563, MSEC-566, MSEC-569, MSEC-571 A, MSEC-571 B, MSEC-577, MSEC-581, MSEC-582A, MSEC-582B, MSEC-584, MSEC-586, MSEC-587, MSEC-588A, MSEC-588B, MSEC-589A, MSEC-589B, MSEC-590, MSEC-594, MSEC-601, MSEC-602A, MS EC-602 B, MSEC-602C, MSEC-602D, MSEC-602 E, MSEC-602F, MSEC-602G, MSEC-602H, MSEC-605, MSEC-607, MSEC-608, MSEC-610, MSEC-611, MSEC-617, MSEC-623, MSEC-625, MSEC-626, MSEC-633, MSEC-638, MSEC-640, MSEC-643, MSEC-652, MSEC-653, MSEC-656A, MSEC-656B, MSEC-658, MSEC-662, MSEC-672, MSEC-673, MSEC-674, MSEC-679A, MSEC-679B, MSEC-681, MSEC-685, MSEC-686, MSEC-689, MSEC-697, MSEC-701, MSEC-714, MSEC-718, MSEC-720, MSEC-721, MSEC-723A, MSEC-723B, MSEC-725A, MSEC-725B, MSEC-725C, MSEC-725D, MSEC-726, MSEC-730, MSEC-732, MSEC-734, MSEC-736, MSEC-745, MSEC-746, MSEC-749, MSEC-754, MSEC-757, MSEC-758, MSEC-764, MSEC-767, MSEC-768, MSEC-770, MSEC-771, MSEC-773, MSEC-778, MSEC-783, MSEC-786, MSEC-793, MSEC-794, MSEC-798, MSEC-804, MSEC-806, MSEC-809, MSEC-812A, MSEC-812B, MSEC-836, MSEC-840A, MSEC-840B, MSEC-855, MSEC-872, MSEC-875, MSEC-879, MSEC-886, MSEC-890, MSEC-892A, MSEC-892B, MSEC-899, MSEC-935, MSEC-952, MSEC-960, MSEC-964, MSEC-966, MSEC-970, MSEC-988, MSEC-995, MSEC-1016, MSEC-1031, MSEC-1204, MSEC-1207, MSEC-1225, MSEC-1240, MSEC-1263B, MSEC-1263C, MSEC-1263D, MSEC-1263E, MSEC-1263F, MSEC-1263G, MSEC-1263H, MSEC-12631, MSEC-1263J, SEC-1263K, MSEC-1263L, MSEC-1263M, MSEC-1263N, MSEC-12630, MSEC-1263P, MSEC-1263S, MSEC-1263T, MSEC-1263U, MSEC-1263V, MSEC-1279, MSEC-1360, MSEC-1370A, MSEC-1370B, MSEC-1465, MSEC-1666, MSEC-1755, MSEC-1263A, or MSEC-1027; or an MSEC having at least 90%, 91%, 92%, 93%, 94%, 95%, 96%, 97%, 98%, or 99% sequence identity to MSEC-74, MSEC-87, MSEC-95, MSEC-118, MSEC-144, MSEC-206A, MSEC-206B, MSEC-212, MSEC-220, MSEC-239, MSEC-259, MSEC-283, MSEC-285, MSEC-288, MSEC-290, MSEC-297A, MSEC-297B, MSEC 297C, MSEC 297D, MSEC 297E, MSEC 297F, MSEC 297G, MSEC 297H, MSEC 2971, MSEC 297J, MSEC 297K, MSEC 297L, MSEC-302A, MSEC-302B, MSEC-302C, MSEC-303, MSEC-304, MSEC-310A, MSEC-31 OB, MSEC 310C, MSEC 310D, MSEC 310E, MSEC 310F, MSEC 310G, MSEC 310H, MSEC 3101, MSEC 310J, MSEC 310K, MSEC 310L, MSEC-315A, MSEC-315B, MSEC-315C, MSEC-318, MSEC-319B, MSEC-320, MSEC-322, MSEC-327, MSEC-330, MSEC-335A, MSEC-335B, MSEC-336A, MSEC-336B, MSEC-336C, MSEC-336D, MSEC-339, MSEC-340, MSEC-341, MSEC-344, MSEC-345, MSEC-346A, MSEC-347, MSEC-348, MSEC-350, MSEC-351, MSEC-352A, MSEC-352B, MSEC-356, MSEC-360A, MSEC-360B, MSEC-361, MSEC-362A, MSEC-362B, MSEC-362C, MSEC-3620, MSEC-362E, MSEC-362F, MSEC-365A, MSEC-365B, MSEC-367, MSEC-378, MSEC-382, MSEC-383, MSEC-384, MSEC-386, MSEC-388, MSEC-393A, MSEC-393B, MSEC-395, MSEC-395B, MSEC-400, MSEC-402, MSEC-403, MSEC-403B, MSEC-405A, MSEC-405B, MSEC-405C, MSEC-406, MSEC-410, MSEC-411, MSEC-413, MSEC-417, MSEC-420A, MSEC-420B, MSEC-421 A, MSEC-421 B, MSEC-421 C, MSEC-4210, MSEC-423, MSEC-425, MSEC-427A, MSEC-427B, MSEC-427C, MSEC-429A, MSEC-429B, MSEC-433, MSEC-438A, MSEC-438B, MSEC-438C, MSEC-438D, MSEC-438E, MSEC-438F, MSEC-438G, MSEC-438H, MSEC-4381, MSEC-439A, MSEC-439B, MSEC-440, MSEC-443, MSEC-444, MSEC-446, MSEC-449, MSEC-450A, MSEC-450B, MSEC-450C, MSEC-450D, MSEC-455A, MSEC-455B, MSEC-455C, MSEC-457, MSEC-461, MSEC-462A, MSEC-462B, MSEC-463, MS EC-464 A, MSEC-464B, MSEC-466, MSEC-472, MSEC-474, MSEC-476, MSEC-476B, MSEC-478A, MSEC-478B, MSEC-478C, MSEC-480, MSEC-481, MSEC-486A, MSEC-486B, MSEC-486C, MSEC-487A, MSEC-487B, MSEC-493A, MSEC-493B, MSEC-495, MSEC-501, MSEC-503, MSEC-508, MSEC-510, MSEC-513, MSEC-517, MSEC-518, MSEC-523, MSEC-527A, MSEC-5276, MSEC-529A, MSEC-529B, MSEC-529C, MSEC-531 A, MSEC-531 B, MSEC-538A, MSEC-538B, MSEC-539A, MSEC-539B, MSEC-540, MSEC-541 A, MSEC-541 B, MSEC-546, MSEC-547, MSEC-550, MSEC-553, MSEC-555, MSEC-556A, MSEC-556B, MSEC-556C, MSEC-563, MSEC-566, MSEC-569, MSEC-571 A, MSEC-571 B, MSEC-577, MSEC-581, MSEC-582A, MSEC-582B, MSEC-584, MSEC-586, MSEC-587, MSEC-588A, MSEC-588B, MSEC-589A, MSEC-589B, MSEC-590, MSEC-594, MSEC-601, MSEC-602A, MSEC-602B, MSEC-602C, MSEC-602D, MSEC-602E, MSEC-602F, MSEC-602G, MSEC-602H, MSEC-605, MSEC-607, MSEC-608, MSEC-610, MSEC-611, MSEC-617, MSEC-623, MSEC-625, MSEC-626, MSEC-633, MSEC-638, MSEC-640, MSEC-643, MSEC-652, MSEC-653, MSEC-656A, MSEC-656B, MSEC-658, MSEC-662, MSEC-672, MSEC-673, MSEC-674, MSEC-679A, MSEC-679B, MSEC-681, MSEC-685, MSEC-686, MSEC-689, MSEC-697, MSEC-701, MSEC-714, MSEC-718, MSEC-720, MSEC-721, MSEC-723A, MSEC-723B, MSEC-725A, MSEC-725B, MSEC-725C, MSEC-7250, MSEC-726, MSEC-730, MSEC-732, MSEC-734, MSEC-736, MSEC-745, MSEC-746, MSEC-749, MSEC-754, MSEC-757, MSEC-758, MSEC-764, MSEC-767, MSEC-768, MSEC-770, MSEC-771, MSEC-773, MSEC-778, MSEC-783, MSEC-786, MSEC-793, MSEC-794, MSEC-798, MSEC-804, MSEC-806, MSEC-809, MSEC-812A, MSEC-812B, MSEC-836, MSEC-840A, MSEC-840B, MSEC-855, MSEC-872, MSEC-875, MSEC-879, MSEC-886, MSEC-890, MSEC-892A, MSEC-892B, MSEC-899, MSEC-935, MSEC-952, MSEC-960, MSEC-964, MSEC-966, MSEC-970, MSEC-988, MSEC-995, MSEC-1016, MSEC-1031, MSEC-1204, MSEC-1207, MSEC-1225, MSEC-1240, MSEC-1263B, MSEC-1263C, MSEC-1263D, MSEC-1263E, MSEC-1263F, MSEC-1263G, MSEC-1263H, MSEC-12631, MSEC-1263J, SEC-1263K, MSEC-1263L, MSEC-1263M, MSEC-1263N, MSEC-12630, MSEC-1263P, MSEC-1263S, MSEC-1263T, MSEC-126311, MSEC-1263V, MSEC-1279, MSEC-1360, MSEC-1370A, MSEC-1370B, MSEC-1465, MSEC-1666, MSEC-1755, MSEC-3363, MSEC-1263A, or MSEC-1027, as described in PCT/US2022/023915, the contents of which are incorporated by reference herein in their entirety.

In some embodiments, the RRM1 and RRM2 sequences for the constructs described herein can be operably coupled to a regulatory cassette comprising the CK8m promoter.

The RRM1 and RRM2 sequences for the constructs described herein can be operably coupled to a regulatory cassette comprising the CK8e regulatory cassette.

### Cardiac Muscle-Specific Promoters

Since transcription from the cardiac troponin T gene (TNNT2) is known to be cardiac-specific (PMID: 26774798), and since studies by other investigators had identified regulatory regions of avian and rodent TNNT2 genes (PMID: 2993302; PMID: 7982978; PMID:18951515, PMID: 9689598) analogous regions of the human TNNT2 gene were cloned. A 495-bp construct (MSEC-495) was tested in fetal rat cardiomyocyte cell cultures and it had high transcriptional activity. MSEC-495 was then miniaturized, to create MSEC-320, and the miniaturized 130-bp enhancer region was then multimerized to create the highly active MSEC-455a. MSEC-455a had high expression in cardiomyocytes and only background expression in non-muscle cells; however it also exhibited expression in skeletal muscle cultures during the onset of differentiation. This behavior is consistent with the fact that an initial step in skeletal muscle differentiation entails activation of numerous cardiac muscle genes, followed by their repression as muscle fibers mature (PMID: 1728592).

### Constitutively Active Promoters

While it can be advantageous to have muscle-specific or cardiac muscle specific expression of ribonucleotide reductase subunit constructs for the generation of dATP donor cells as described herein, when the donor cell is not a muscle cell or a cardiomyocyte, it can be effective to drive expression of the ribonucleotide reductase subunits from a non-muscle-specific promoter or regulatory element. Thus, in some embodiments, the ribonucleotide reductase expression cassette is operably linked to a constitutively active regulatory element or promoter.

A constitutively active promoter is one that is active in all cell types of a given organism and not substantially regulated up or down according to cell type. Such promoters carry out the transcription of operatively linked genes continuously in the cell.

Examples for constitutively active promoters include, but are not limited to the CMV, Thymidine Kinase, SV40, EF1A, and CAG promoters, sequences for which are set out below. In some embodiments, variants of these that drive expression include sequences that are at least 85%, at least 90%, at least 95%, at least 96%, at least 97%, at least 98%, or at least 99% identical to the promoter sequence.

One who is skilled in the art can make and use the CMV promoter (see, e.g., GenBank X96612.1, gene=“CMV”, regulatory_class=“promoter” and Uetz, P. and Zeller, R. Vectors for expression of protein-A-tagged proteins in vertebrate cells. Anal. Biochem. 237 (1), 161-163 (1996)).

One who is skilled in the art can make and use the Herpes Simplex Virus (HSV) thymidine kinase (TK) promoter (see, e.g., GenBank L37440.1, 36.303, regulatory_class=“promoter”, note=“herpes simplex virus thymidine kinase (HSV TK) promoter and viral Polyoma Py F441 enhancer”, GenBank M10409.1, and ElKareh, A. et al. ‘Transactivation’ control signals in the promoter of the herpesvirus thymidine kinase gene. Proc. Natl. Acad. Sci. U.S.A. 82 (4), 1002-1006 (1985)).

One who is skilled in the art can make and use the SV40 promoter (see, e.g., GenBank x96612.1, gene=“SV40”, regulatory_class=“promoter” and Uetz, P. and Zeller, R. Vectors for expression of protein-A-tagged proteins in vertebrate cells. Anal. Biochem. 237 (1), 161-163 (1996)).

One who is skilled in the art can make and use the EF1a promoter (see, e.g., GenBank EU424173.1, complement (3695 . . . 4862), regulatory_class=“promoter”, note=“EF1-alpha (elongation factor 1 alpha) promoter and first intron”, GenBank MG520662.1, 1.1179, regulatory_class=“promoter”, note=“ef1-alpha promoter”, and Kyriakakis, P. et al. Biosynthesis of Orthogonal Molecules Using Ferredoxin and Ferredoxin-NADP+ Reductase Systems Enables Genetically Encoded PhyB Optogenetics. ACS Synth Biol (2018).

One who is skilled in the art can make and use the CAG promoter, which is also known as a hybrid construct consisting of the cytomegalovirus (CMV) enhancer fused to the chicken beta-actin promoter (see e.g., GenBank LC744045.1, regulatory 4853 . . . 5232 and 5234 . . . 5511 and GenBank MT276428.1, misc_feature 22 . . . 1754).

### AAV Vectors

While other transfection methods are applicable for cells transformed in vitro, in some embodiments, a ribonucleotide reductase expression cassette can be introduced to a cell using a viral vector, e.g., an AAV vector. AAV is a parvovirus which belongs to the genus Dependoparvovirus. The AAV genome is a linear, single-stranded DNA molecule containing 4681 nucleotides. The AAV genome generally comprises an internal non-repeating genome flanked on each end by inverted terminal repeats (ITRs). The ITRs are approximately 145 base pairs (bp) in length. The ITRs have multiple functions, including as origins of DNA replication and as packaging signals for the viral genome.

As used herein, the term “AAV vector” refers to a vector derived from an adeno-associated virus serotype, including without limitation, AAV1, AAV2, AAV3, AAV4, AAV5, AAV6, AAV7, AAV8, and AAV9. AAV vectors can have one or more of the AAV wild-type genes deleted in whole or part, e.g., the rep and/or cap genes, but retain functional flanking ITR sequences. Functional ITR sequences are necessary for the rescue, replication, and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

A “recombinant AAV vector” or “rAAV vector” comprises an infectious, replication-defective virus composed of an AAV protein shell encapsulating a heterologous nucleotide sequence of interest that is flanked on both sides by AAV ITRs. An rAAV vector is produced in a suitable host cell comprising an AAV vector, AAV helper functions, and accessory functions. In this manner, the host cell is rendered capable of encoding AAV polypeptides that are required for packaging the AAV vector (containing a recombinant nucleotide sequence of interest) into infectious recombinant virion particles for subsequent gene delivery.

In various embodiments, the delivery vehicle can comprise an adeno-associated virus (AAV) vector or a recombinant adeno-associated virus (rAAV) vector. The AAV vector can be a serotype 6 AAV (AAV6). Likewise, the rAAV vector may be a serotype 6 rAAV (rAAV6). The AAV vector can be a serotype 8 AAV (AAV8). Likewise, the rAAV vector can be a serotype 8 rAAV (rAAV8). The AAV vector can be a serotype 9 AAV (AAV9). Likewise, the rAAV vector can be a serotype 9 rAAV (rAAV9). The rAAV vector can be comprised of AAV2 genomic inverted terminal repeat (ITR) sequences pseudotyped with capsid proteins derived from AAV serotype 6 (rAAV2/6). Other suitable serotypes of the AAV or rAAV known in the art can be used. AAV6 is particularly attractive due to efficient infection and transduction of muscle cells, including cardiac muscle cells.

2-Deoxy-ATP (2-Deoxyadenosine Triphosphate; dATP)

Deoxyadenosine is a derivative of the nucleoside adenosine. It is composed of adenine covalently attached to a deoxyribose moiety via an N9-glycosidic bond. Deoxyribose differs from ribose by the absence of oxygen in the 3′ position of its ribose ring. Deoxyadenosine is a critical component of DNA; deoxyadenosine is the DNA nucleoside A, which pairs with deoxythymidine (T) in double-stranded DNA.

dATP is produced in the cell by the action of ribonucleotide reductase, which comprises RRM1 and RRM2 subunits. An increase in ribonucleotide reductase activity in a cell can therefore increase dATP production in the cell. An increase in RRM1 and RRM2 expression in a cell, e.g., from a cassette as described herein, can thus increase dATP production in the cell. However, where RRM2 can be less stable than RRM1 in the cell, an increase in RRM2 expression, stability or both can also increase ribonucleotide reductase activity. In one embodiment, cellular expression of an isolated rrm2 nucleic acid or an Rrm2 polypeptide encoded by the nucleic acid, or a composition thereof, increases dATP in the cell. For example, cellular expression of an isolated rrm2 nucleic acid or an Rrm2 polypeptide encoded by it, or a composition thereof increases intracellular dATP levels at least 10%, at least 20%, at least 30%, at least 40%, at least 50%, at least 60%, at least 70%, at least 80%, at least 90%, at least 95%, at least 99% or more, or at least 1-fold, at least 2-fold, at least 3-fold, at least 4-fold, at least 5-fold, at least 6-fold, at least 7-fold, at least 8-fold, at least 9-fold, at least 10-fold or more as compared to expression of wild-type Rrm2. Deoxyadenosine levels in a cell, such as a cell expressing RRM1 and RRM2 from a cassette as described herein, expressing RRM1 and a modified or stabilized RRM2 from a cassette as described herein, or expressing a modified or stabilized RRM2 from a cassette as described herein can be measured via e.g., HPLC or mass spectrometry using standard protocols.

### Compositions

Various aspects described herein provide a composition comprising, consisting of, or consisting essentially of any of the isolated nucleic acids, vectors, polypeptides, RNR complexes or cells including them as described herein.

One aspect provided herein is a pharmaceutical composition comprising, consisting of, or consisting essentially of any of the dATP donor cells, isolated nucleic acids, vectors, polypeptides, or RNR complexes described herein. As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier in which the active ingredient would not be found to occur in nature.

Pharmaceutically acceptable or physiologically tolerable materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically, such compositions are prepared as injectable either as liquid solutions or suspensions, however, solid, including, but not limited to, e.g., lyophilized, forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present technology can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used with the methods described herein that will be effective in the treatment of a particular disorder or condition will depend on the nature of the disorder or condition, and can be determined by standard clinical techniques.

### Administration of Cells

As used herein, the terms “administering,” “introducing”, “engraftment”, and “transplanting” are used interchangeably in the context of the placement of cells, e.g. dATP donor cells, as described herein into a subject, by a method or route which results in at least partial localization of the introduced cells at a desired site, such as a site of injury or repair, such that a desired effect(s) is produced. The dATP donor cells can be implanted directly to the heart, or alternatively be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. As one of skill in the art will appreciate, long-term engraftment of the cardiomyocytes is desired as cardiomyocytes do not proliferate to an extent that the heart can heal from an acute injury comprising cardiomyocyte death. Thus, a graft can be used to replace lost cells that occur during injury. In other embodiments, the cells can be administered via an indirect systemic route of administration, such as an intraperitoneal or intravenous route. It is contemplated, for example, that RNR-cassette modified, in vitro-differentiated macrophages could be administered systemically and permitted to home to the injured heart tissue. Where the donor cells are in vitro-differentiated cardiomyocytes or cardiomyocyte progenitors, methods for improving engraftment or preventing engraftment arrhythmias, such as those described in e.g., US 2020-0085880, WO 2020/190739, or WO 2021/163037, the contents of each of which are incorporated herein by reference in their entirety, can be combined with the methods and compositions described herein.

When provided prophylactically, the dATP donor cells can be administered to a subject, for example, to avoid heart failure due to prior myocardial infarction or left ventricular insufficiency, congestive heart failure etc. Accordingly, the prophylactic administration of a population of dATP donor cells serves to prevent a heart failure disorder or maladaptive cardiac remodeling. In some embodiments, the dATP donor cells can be used to improve the function of a normal heart.

The cells described herein can be administered to the heart in an effective amount for the treatment of a cardiac disease or disorder. The term “effective amount” as used herein refers to the amount of a population of dATP donor cells needed to alleviate at least one or more symptoms of a disease or disorder, including but not limited to a cardiac injury or a cardiac disease or disorder. An “effective amount” relates to a sufficient amount of a composition to provide the desired effect, e.g., treat a subject having an infarct zone following myocardial infarction, prevent onset of heart failure following MI or cardiac injury etc. The term “therapeutically effective amount” therefore refers to an amount of human dATP donor cells or a composition such cells that is sufficient to promote a particular effect when administered to atypical subject, such as one who has, or is at risk for, a cardiac disease or disorder. An effective amount as used herein would also include an amount sufficient to prevent or delay the development of a symptom of the disease, alter the course of a disease symptom (for example but not limited to, slow the progression of a symptom of the disease), or reverse a symptom of the disease. It is understood that for any given case, an appropriate “effective amount” can be determined by one of ordinary skill in the art using routine experimentation.

In some embodiments, the subject is first diagnosed as having a disease or disorder affecting the myocardium prior to administering the cells according to the methods described herein. In some embodiments, the subject is first diagnosed as being at risk of developing cardiac disease (e.g., heart failure following myocardial injury) or disorder prior to administering the cells.

For use in the various aspects described herein, an effective amount of dATP donor cells, comprises at least 1×10³, at least 1×10⁴, at least 1×10⁵, at least 5×10⁵, at least 1×10⁶, at least 2×10⁶, at least 3×10⁶, at least 4×10⁶, at least 5×10⁶, at least 6×10⁶, at least 7×10⁶, at least 8×10⁶, at least 9×10⁶, at least 1×10⁷, at least 1.1×10⁷, at least 1.2×10⁷, at least 1.3×10⁷, at least 1.4×10⁷, at least 1.5×10⁷, at least 1.6×10⁷, at least 1.7×10⁷, at least 1.8×10⁷, at least 1.9×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 5×10⁸, at least 7×10⁸, at least 1×10⁹, at least 2×10⁹, at least 3×10⁹, at least 4×10⁹, at least 5×10⁹ or more dATP donor cells.

The dATP donor cells can be derived from one or more donors, or can be obtained from an autologous source. In some embodiments of the aspects described herein, the donor cells are expanded or differentiated from progenitor cells in culture prior to administration to a subject in need thereof.

Exemplary modes of administration for use in the methods described herein include, but are not limited to, injection, intracardiac delivery, systemic administration and implantation (with or without a scaffold material). “Injection” includes, without limitation, intracardiac, intravenous, intramuscular, intraarterial, intradermal, intraperitoneal and subcutaneous.

In some embodiments, a therapeutically effective amount of dATP donor cells is administered using direct injection into the heart including, but not limited to administration during open-heart surgery or by intracardiac injection through an intact chest, or by intramyocardial delivery using an injection catheter or other device. In some aspects of these methods, a therapeutically effective amount of dATP donor cells are administered using a systemic route, such as an intraperitoneal or intravenous route. In some aspects of these methods, a therapeutically effective amount of dATP donor cells is not administered using systemic or intraperitoneal administration. These methods are particularly aimed at therapeutic and prophylactic treatments of human subjects having, or at risk of having, a cardiac disease or disorder. The dATP donor cells described herein can be administered to a subject having any cardiac disease or disorder by any appropriate route which results in an effective treatment in the subject. In some embodiments of the aspects described herein, a subject having a cardiac disorder is first selected prior to administration of the cells.

In some embodiments, an effective amount of dATP donor cells are administered to a subject by intracardiac administration or delivery. As defined herein, “intracardiac” administration or delivery refers to all routes of administration whereby a population of dATP donor cells is administered in a way that results in direct contact of these cells with the myocardium of a subject, including, but not limited to, direct cardiac injection, intra-myocardial injection(s), intra-infarct zone injection, injection during surgery (e.g., cardiac bypass surgery, during implantation of a cardiac mini-pump or a pacemaker, etc.). In some such embodiments, the cells are injected into the myocardium, or into the cavity of the atria and/or ventricles. In some embodiments, intracardiac delivery of cells includes administration methods whereby cells are administered, for example as a cell suspension, to a subject undergoing surgery via a single injection or multiple “mini” injections into the desired region of the heart.

The choice of formulation for a cell composition will depend upon the specific composition used and the number of dATP donor cells to be administered; such formulations can be adjusted by the skilled practitioner. However, as an example, where the composition is donor dATP cells in a pharmaceutically acceptable carrier, the composition can be a suspension of the cells in an appropriate buffer (e.g., saline buffer) at an effective concentration of cells per mL of solution. Formulations can also include one or more agents to enhance retention, viability, proliferation and/or vascularization, and can include, for example, protein- and non-protein hydrogels. The formulation can also include cell nutrients, a simple sugar (e.g., for osmotic pressure regulation) or other components to maintain the viability of the cells. Alternatively, the formulation can comprise a scaffold, such as a biodegradable scaffold.

In some embodiments, additional agents to aid in treatment of the subject can be administered before or following treatment with the cells as described herein. Such additional agents can be used to prepare the target tissue for administration of the cells. Alternatively, the additional agents can be administered after the cell transplantation to support the engraftment and growth of the administered cells into the heart. In some embodiments, the additional agent comprises growth factors, such as VEGF or PDGF. Other exemplary agents can be used to reduce the load on the heart while the dATP donor cells are engrafting (e.g., beta blockers, medications to lower blood pressure etc.).

### Efficacy

The efficacy of a given treatment for improving fractional shortening, ejection fraction or other measures of cardiac contractility in vivo can be determined by the skilled clinician. However, a treatment is considered “effective treatment,” as the term is used herein, if any one or all of the signs or symptoms of impaired contractility is/are altered in a beneficial manner, or other clinically accepted symptoms or markers of disease are improved, or ameliorated, e.g., by at least 10% following treatment with a e.g., a composition comprising dATP donor cells. Efficacy can also be measured by failure of an individual to worsen as assessed by stabilization of the disease, or the reduced need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the graft rejection or (2) relieving the disease, e.g., causing regression of symptoms; and (3) preventing or reducing the likelihood of the development of the disease, or preventing secondary diseases/disorders (e.g., pulmonary or systemic edema, pneumonia etc).

An effective amount for the treatment of a disease means that amount which, when administered to a mammal in need thereof, is sufficient to result in effective treatment as that term is defined herein, for that disease. Efficacy of an agent can be determined by assessing a reduction in one or more physical indicators of the disease, such as e.g., lung congestion, edema, shortness of breath, rapid heart rate, chest pain, fainting etc.

Exemplary indicators of cardiac disease or cardiac disorder, or cardiac injury can be monitored to determine the efficacy of treatment. Non-limiting examples of such functional indicators or parameters, e.g., stroke volume, heart rate, left ventricular ejection fraction, heart rate, heart rhythm, blood pressure, heart volume, regurgitation, etc. as well as biochemical indicators, such as a decrease in markers of cardiac injury, such as serum lactate dehydrogenase, or serum troponin, as well as a decrease in markers indicating cardiac failure such as B-type natriuretic peptide (BNP), among others. As one example, myocardial ischemia and reperfusion are associated with reduced cardiac function. Subjects that have suffered an ischemic cardiac event and/or that have received reperfusion therapy have reduced cardiac function when compared to that before ischemia and/or reperfusion. Measures of cardiac function include, for example, ejection fraction and fractional shortening. Ejection fraction is the fraction of blood pumped out of a ventricle with each heartbeat. The term ejection fraction applies to both the right and left ventricles. LVEF refers to the left ventricular ejection fraction (LVEF). Fractional shortening refers to the difference between end-diastolic and end-systolic dimensions divided by end-diastolic dimension.

Non-limiting examples of clinical tests that can be used to assess cardiac functional parameters include echocardiography (with or without Doppler flow imaging), cardiac magnetic resonance imaging, electrocardiogram (EKG), exercise stress test, Holter monitoring, or measurement of B-natriuretic peptide.

Where necessary or desired, animal models of cardiac injury or cardiac disease can be used to gauge the effectiveness of a particular composition as described herein. For example, an isolated working rabbit or rat heart model, or a coronary ligation model in small animals (mouse, rat, rabbit) or large animals (canines, porcines, non-human primates) can be used. Animal models of cardiac function are useful for monitoring infarct zones, coronary perfusion, electrical conduction, left ventricular end diastolic pressure, left ventricular ejection fraction, heart rate, blood pressure, degree of hypertrophy, diastolic relaxation function, cardiac output, heart rate variability, and ventricular wall thickness, etc.

### EXAMPLES

Cell-based therapies are mostly used as a means of replacing tissue, delivering paracrine factors, or improving ventricular wall mechanics. The approach described herein can provide continual inotropic support through dATP delivery from grafted hiPSC-CMRNR (human induced pluripotent stem cell derived cardiomyocytes expressing RNR) to coupled host myocardium. This strategy can restore lost contractile function in addition to preventing future decline. These methods should improve the oxygenated blood to the heart, stabilizing the decline in heart failure (HF) with little or no metabolic consequences.

### Example 1: HiPSCs-Derived Cardiomyocytes Overexpressing deoxyATP to Restore Cardiac Function

In one aspect, a goal of the methods and compositions described herein is to provide clinical treatments by directing human stem cells to regenerate damaged tissue and rescue lost contractility in the setting of chronic myocardial infarction. The approach described herein comprises genetically engineering human induced pluripotent stem cell-derived cardiomyocytes to express high levels of deoxyATP (dATP) for remuscularization as well as improved function.

dATP is an endogenous activator of cardiac myosin and it improves contractility with no adverse effect on cardiac relaxation. It has been shown that dATP pushes myosin heads to the disordered relaxed state (DRX), which has faster ATPase turnover. dATP is synthesized endogenously by ribonucleotide reductase (RNR), which is composed of two different subunits (R1 and R2). R2 is normally proteolytically downregulated in cardiac myocytes.

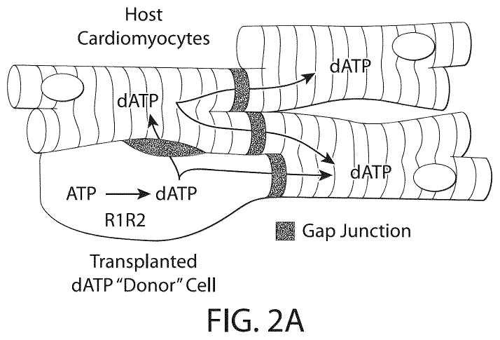

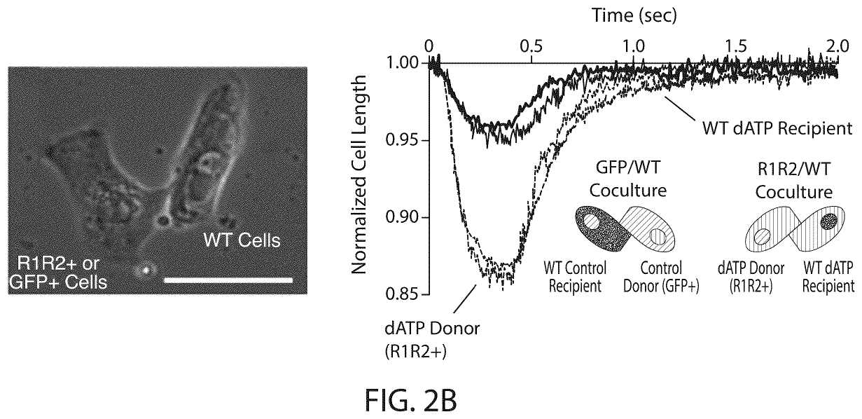

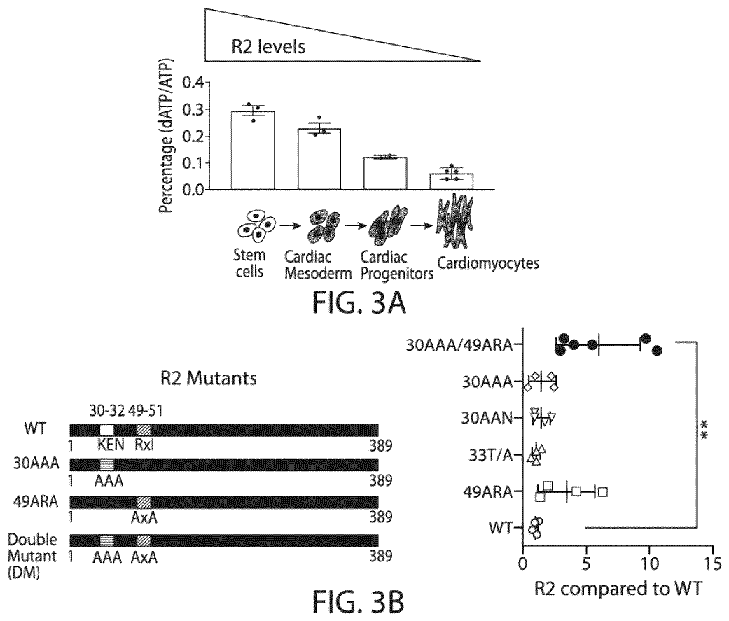

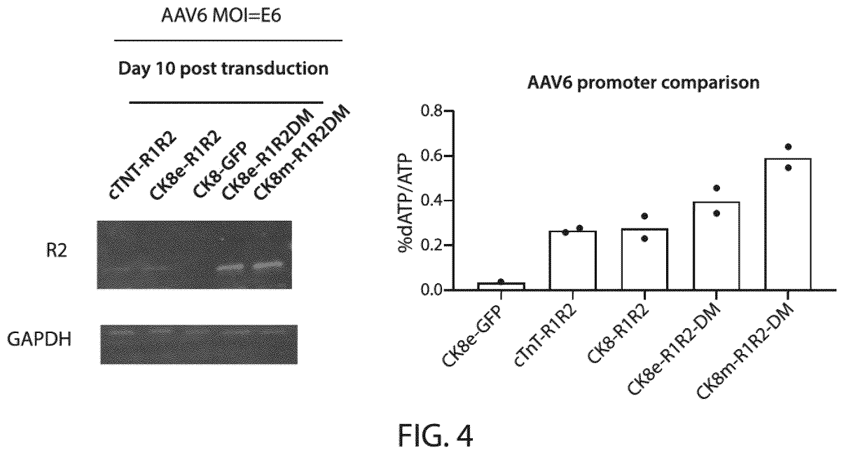

The inventors' previous research showed that viral vector based RNR overexpression improves cardiac function in small and large animal models of myocardial infarction. FIGS. 2A and 2B shows data indicating that dATP diffuses via gap junctions to neighboring cardiomyocytes. FIG. 3A shows decreasing R2 levels as stem cells differentiate into cardiomyocytes. FIG. 3B and FIG. 4 show elevation of RNR expression in cardiomyocytes by directed mutagenesis of the R2 subunit.

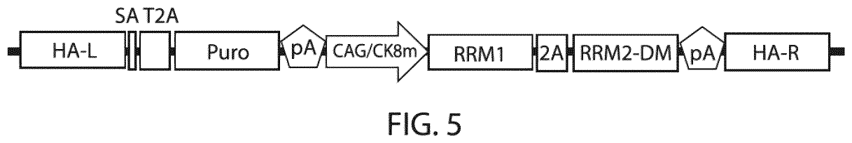

The inventors also conducted a study of RNR expression and dATP levels using different promoters (see FIG. 4). Analysis of different promoters (as shown in an exemplary schematic in FIG. 5) indicated that CK8m expressing the R1 and double-mutated (DM) R2 subunit produced the highest levels of dATP in adenovirally transduced cells.

As described herein, (i) dATP-CMs can remuscularize and improve functions in the chronically failing heart, (ii) dATP-CMs, e.g., CK8m-dATP-CMs, can engraft and improve function in uninjured heart—significantly higher levels of dATP (vs cells with CAG-driven RNR expression) can promote its faster diffusion and improvement in function, (iii) CK8m-dATP-CMs (vs CAG) can survive transplantation in a chronically injured heart, and (iv) dATP-CMs, e.g., CK8m-dATP-CMs can improve function in a chronically injured heart at, e.g., 1-month and 3-month timepoints.

1. A dATP donor cell comprising a nucleic acid sequence encoding a ribonucleotide reductase expression cassette operably linked to a muscle-specific promoter.

2. The dATP donor cell of claim 1, wherein the cell is an induced pluripotent stem cell or an embryonic stem cell; and/or wherein the cell is a human cell.

3. (canceled)

4. The dATP donor cell of claim 2, wherein the cell is capable of forming gap junctions with a cardiomyocyte, and the cell comprises a cardiomyocyte, a cardiac progenitor cell, a fibroblast, a mesenchymal cell, a smooth muscle cell, an endothelial cell, or a hematopoietic cell.

5. (canceled)

6. The dATP donor cell of claim 4, wherein the cell is a cardiac progenitor cell or a cardiomyocyte, and wherein the cardiac progenitor cell or cardiomyocyte are in vitro-differentiated.

7. (canceled)

8. The dATP donor cell of claim 1, wherein the ribonucleotide reductase expression cassette comprises a sequence encoding a ribonucleotide reductase catalytic subunit (RRM1) and a ribonucleotide reductase regulatory subunit (RRM2).

9. The dATP donor cell of claim 8, wherein the RRM2 subunit comprises a double mutation and has increased stability compared to the wild-type RRM2 subunit.

10. The dATP donor cell of claim 9, wherein the double mutation comprises 30AAA/49ARA mutations in SEQ ID NO. 26.

11. The dATP donor cell of claim 8, wherein the sequence of the RRM1 subunit is SEQ ID NO: 1, and the sequence of the RRM2 subunit comprises SEQ ID NO: 3.

12. The dATP donor cell of claim 1, wherein the muscle-specific promoter is CK8m or CK8e.

13. (canceled)

14. The dATP donor cell of claim 12, wherein the muscle-specific promoter is a cardiac muscle-specific promoter selected from the group consisting of: MSEC-320, MSEC-455c, MSEC-455a, MSEC-571, MSEC-725a, MSEC-875, and CK8m.

15. (canceled)

16. A dATP donor cell comprising a nucleic acid sequence encoding a ribonucleotide reductase expression cassette operably linked to a constitutively active promoter.

17. The dATP donor cell of claim 16, wherein the constitutively active promoter is selected from CMV, Thymidine Kinase, SV40, EF1A, and CAG promoters.

18. The dATP donor cell of claim 16, wherein the cell is an induced pluripotent stem cell or an embryonic stem cell.

19. (canceled)

20. The dATP donor cell of claim 18 wherein the cell is capable of forming gap junctions with a cardiomyocyte, and the cell comprises a cardiomyocyte, a cardiac progenitor cell, a fibroblast, a mesenchymal cell, a smooth muscle cell, an endothelial cell, or a hematopoietic cell.

21. The dATP donor cell of claim 20, wherein the cell is a cardiac progenitor cell or a cardiomyocyte, and wherein the cardiac progenitor cell or cardiomyocyte are in vitro-differentiated.

22. (canceled)

23. The dATP donor cell of claim 16, wherein the cell is a human cell.

24. The dATP donor cell of claim 16, wherein the ribonucleotide reductase expression cassette comprises sequence encoding a ribonucleotide reductase catalytic subunit (RRM1) and a ribonucleotide reductase regulatory subunit (RRM2).

25. The dATP donor cell of claim 24, wherein the RRM2 subunit comprises a double mutation and has increased stability compared to the wild-type RRM2 subunit.

26. The dATP donor cell of claim 25, wherein the double mutation comprises 30AAA/49ARA mutations in SEQ ID NO. 26.

27. The dATP donor cell of claim 24, wherein the sequence of the RRM1 subunit is SEQ ID NO: 1, and the sequence of the RRM2 subunit comprises SEQ ID NO: 3.

28-74. (canceled)


SEQUENCE LISTING (26 sequences)

SEQ ID NO: 1 - RNA, 2379 nt, Homo sapiens
  source 1..2379: mol_type = mRNA
atgcatgtgatcaagcgagatggccgccaagaacgagtcatgtttgacaaaattacatctcgaatccagaagctttgttatggactcaatatggattttgttgatcctgctcagatcaccatgaaagtaatccaaggcttgtacagtggggtcaccacagtggaactagatactttggctgctgaaacagctgcaaccttgactactaagcaccctgactatgctatcctggcagccaggatcgctgtctctaacttgcacaaagaaacaaagaaagtgttcagtgatgtgatggaagacctctataactacataaatccacataatggcaaacactctcccatggtggccaagtcaacattggatattgttctggccaataaagatcgcctgaattctgctattatctatgaccgagatttctcttacaattacttcggctttaagacgctagagcggtcttatttgttgaagatcaatggaaaagtggctgaaagaccacaacatatgttgatgagagtatctgttgggatccacaaagaagacattgatgcagcaattgaaacatataatcttctttctgagaggtggtttactcatgcttcgcccactctcttcaatgctggtaccaaccgcccacaactttctagctgttttcttctgagtatgaaagatgacagcattgaaggcatttatgacactctaaagcaatgtgcattgatttctaagtctgctggaggaattggtgttgctgtgagttgtattcgggctactggcagctacattgctgggactaatggcaattccaatggccttgtaccgatgctgagagtatataacaacacagctcgatatgtggatcaaggtgggaacaagcgtcctggggcatttgctatttacctggagccttggcatttagacatctttgaattccttgatttaaagaagaacacaggaaaggaagagcagcgtgccagagatcttttctttgctctttggattccggatctcttcatgaaacgagtggagactaatcaggactggtctttgatgtgtccaaatgagtgtcctggtctggatgaggtttggggagaggaatttgagaaactatatgcaagttatgagaaacaaggtcgtgtccgcaaagttgtaaaagctcagcagctttggtatgccatcattgagtctcagacggaaacaggcaccccgtatatgctctacaaagattcctgtaatcgaaagagcaaccagcagaacctgggaaccatcaaatgcagcaacctgtgcacagaaatagtggagtacaccagcaaagatgaggttgctgtttgtaatttggcttccctggccctgaatatgtatgtcacatcagaacacacatacgactttaagaagttggctgaagtcactaaagtcgttgtccgaaacttgaataaaattattgatataaactactatcctgtaccagaggcatgcctatcaaataaacgccatcgccccattggaattggggtacaaggtctggcagatgcttttatcctgatgagatacccttttgagagtgcagaagcccagttactgaataagcagatctttgaaactatttattatggtgctctggaagccagctgtgaccttgccaaggagcagggcccatacgaaacctatgagggctctccagttagcaaaggaattcttcagtatgatatgtggaatgttactcctacagacctatgggactggaaggttctcaaggagaagattgcaaagtatggtataagaaacagtttacttattgccccgatgcctacagcttccactgctcagatcctggggaataatgagtccattgaaccttacaccagcaacatctatactcgcagagtcttgtcaggagaatttcagattgtaaatcctcacttattgaaagatcttaccgagcggggcctatggcatgaagagatgaaaaaccagattattgcatgcaatggctctattcagagcataccagaaattcctgatgacctgaagcaactttataaaactgtgtgggaaatctctcagaaaactgttctcaagatggcagctgagagaggtgctttcattgatcaaagccaatctttgaacatccacattgctgagcctaactatggcaaactcactagtatgcacttctacggctggaagcagggtttgaagactgggatgtattatttaaggacaagaccagcggctaatccaatccagttcactctaaataaggagaagctaaaagataaagaaaaggtatcaaaagaggaagaagagaaggagaggaacacagcagccatggtgtgctctttggagaatagagatgaatgtctgatgtgtggatcctga

SEQ ID NO: 2 - protein, 792 aa, Homo sapiens
MHVIKRDGRQERVMFDKITSRIQKLCYGLNMDFVDPAQITMKVIQGLYSGVTTVELDTLAAETAATLTTKHPDYAILAARIAVSNLHKETKKVFSDVMEDLYNYINPHNGKHSPMVAKSTLDIVLANKDRLNSAIIYDRDFSYNYFGFKTLERSYLLKINGKVAERPQHMLMRVSVGIHKEDIDAAIETYNLLSERWFTHASPTLFNAGTNRPQLSSCFLLSMKDDSIEGIYDTLKQCALISKSAGGIGVAVSCIRATGSYIAGTNGNSNGLVPMLRVYNNTARYVDQGGNKRPGAFAIYLEPWHLDIFEFLDLKKNTGKEEQRARDLFFALWIPDLFMKRVETNQDWSLMCPNECPGLDEVWGEEFEKLYASYEKQGRVRKVVKAQQLWYAIIESQTETGTPYMLYKDSCNRKSNQQNLGTIKCSNLCTEIVEYTSKDEVAVCNLASLALNMYVTSEHTYDFKKLAEVTKVVVRNLNKIIDINYYPVPEACLSNKRHRPIGIGVQGLADAFILMRYPFESAEAQLLNKQIFETIYYGALEASCDLAKEQGPYETYEGSPVSKGILQYDMWNVTPTDLWDWKVLKEKIAKYGIRNSLLIAPMPTASTAQILGNNESIEPYTSNIYTRRVLSGEFQIVNPHLLKDLTERGLWHEEMKNQIIACNGSIQSIPEIPDDLKQLYKTVWEISQKTVLKMAAERGAFIDQSQSLNIHIAEPNYGKLTSMHFYGWKQGLKTGMYYLRTRPAANPIQFTLNKEKLKDKEKVSKEEEEKERNTAAMVCSLENRDECLMCGS

SEQ ID NO: 3 - RNA, 1170 nt, Homo sapiens
  source 1..1170: mol_type = mRNA
atgctctccctccgtgtcccgctcgcgcccatcacggacccgcagcagctgcagctctcgccgctgaaggggctcagcttggtcgacaaggagaacacgccgccggccctgagcgggacccgcgtcctggccagcaagaccgcgaggaggatcttccaggagcccacggagccgaaaactaaagcagctgcccccggcgtggaggatgagccgctgctgagagaaaacccccgccgctttgtcatcttccccatcgagtaccatgatatctggcagatgtataagaaggcagaggcttccttttggaccgccgaggaggtggacctctccaaggacattcagcactgggaatccctgaaacccgaggagagatattttatatcccatgttctggctttctttgcagcaagcgatggcatagtaaatgaaaacttggtggagcgatttagccaagaagttcagattacagaagcccgctgtttctatggcttccaaattgccatggaaaacatacattctgaaatgtatagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggttttcagagtagagaacccatttgactttatggagaatatttcactggaaggaaagactaacttctttgagaagagagtaggcgagtatcagaggatgggagtgatgtcaagtccaacagagaattcttttaccttggatgctgacttctaa

SEQ ID NO: 4 - protein, 389 aa, Homo sapiens
MLSLRVPLAPITDPQQLQLSPLKGLSLVDKENTPPALSGTRVLASKTARRIFQEPTEPKTKAAAPGVEDEPLLRENPRRFVIFPIEYHDIWQMYKKAEASFWTAEEVDLSKDIQHWESLKPEERYFISHVLAFFAASDGIVNENLVERFSQEVQITEARCFYGFQIAMENIHSEMYSLLIDTYIKDPKEREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEYQRMGVMSSPTENSFTLDADF

SEQ ID NO: 5 - RNA, 1170 nt, synthetic construct
  source 1..1170: mol_type = other RNA
atgctgagtctgagggtcccactggcacctatcaccgatccacagcagctgcagctgagcccactgaaaggcctgagtctggtcgataaagagaacacaccacctgcactgagtggcactcgggtgctggcatcaaagaccgcccggagaattttccaggagccaaccgaacccaaaacaaaggccgctgcacctggggtcgaggacgaaccactgctgagagagaatcccaggcgcttcgtgatttttcctatcgaataccacgatatttggcagatgtataagaaagctgaggcaagtttctggacagctgaggaagtggacctgagcaaagacatccagcactgggaatccctgaagccagaggaaaggtacttcatttctcatgtgctggcattctttgccgctagtgacgggatcgtgaacgagaatctggtcgaacgctttagccaggaggtgcagatcactgaagcccgatgcttctatggatttcagattgctatggagaacatccattcagaaatgtacagcctgctgattgacacctatatcaaagatcctaaggagcgcgagttcctgtttaatgccattgagacaatgccatgtgtgaagaaaaaggcagactgggctctgcgatggatcggcgataaggaggctacttacggggaaagagtggtcgcattcgcagccgtggagggaattttcttttctggcagtttcgcttccatcttttggctgaaaaagcgaggcctgatgcctgggctgaccttttccaacgagctgatttctcgcgacgaaggcctgcactgcgatttcgcctgtctgatgtttaaacacctggtgcataagccctctgaggaacgagtccgggagatcattatcaacgcagtgaggatcgagcaggagttcctgacagaagccctgcctgtcaaactgattggcatgaattgcactctgatgaagcagtacatcgagtttgtggccgacaggctgatgctggaactgggattctcaaaggtgtttcgcgtcgagaacccattcgattttatggagaatatcagcctggaaggcaaaacaaacttctttgagaagagagtcggggaatatcagaggatgggcgtgatgagcagccccactgagaatagcttcaccctggacgccgatttttga

SEQ ID NO: 6 - RNA, 3633 nt, synthetic construct
  source 1..3633: mol_type = other RNA
gctagcgaattcgccaccatgcacgtcatcaagagagacgggaggcaggaaagagtcatgttcgataaaatcacttcaagaatccagaaactgtgttacgggctgaacatggacttcgtcgatcctgcccagattaccatgaaagtgatccagggactgtactctggcgtcaccacagtggagctggacacactggccgctgaaaccgcagccacactgactaccaaacacccagattatgcaattctggctgcacggatcgccgtgagtaatctgcataaggagacaaagaaagtcttctcagacgtgatggaggacctgtacaattatatcaaccctcacaatgggaaacattcaccaatggtcgctaagagcactctggacattgtgctggccaacaaagatcggctgaacagcgctatcatctacgaccgggatttcagttacaactacttcggctttaagacactggagagatcatatctgctgaaaatcaatgggaaggtggccgaacggcctcagcacatgctgatgagagtcagcgtgggcattcataaggaggacattgatgccgctatcgaaacttacaacctgctgagcgagcgctggttcacccacgcttcccctacactgtttaacgcaggaaccaatcgaccacagctgagcagctgcttcctgctgagcatgaaggacgattccatcgagggcatctacgacaccctgaaacagtgcgcactgatttctaagagtgccggcgggatcggagtcgctgtgagttgtattcgggcaaccggctcatatatcgccggcacaaacggcaacagcaacgggctggtccccatgctgagggtgtacaacaatacagcccgctatgtggatcagggaggcaacaagagaccaggagcatttgccatctacctggaaccctggcacctggacattttcgagtttctggatctgaagaaaaatactggcaaagaggaacagagggctcgcgacctgttctttgcactgtggattcccgacctgttcatgaagagggtggagaccaaccaggactggagcctgatgtgccccaatgagtgtcctgggctggatgaagtgtggggagaggaatttgaaaaactgtacgccagttatgagaagcagggccgagtgcggaaagtggtcaaggcccagcagctgtggtacgctatcattgagagccagacagaaactggcaccccctacatgctgtataaagactcttgcaaccgcaagagtaaccagcagaatctggggaccatcaaatgcagcaatctgtgtacagagattgtggaatatacttccaaggatgaggtcgccgtgtgtaacctggcatcactggccctgaatatgtacgtcacaagcgagcacacttatgacttcaagaaactggctgaagtgaccaaagtggtcgtgaggaatctgaacaagatcattgacatcaactactatcccgtgcctgaggcctgcctgagcaataagagacataggcccatcgggattggagtgcagggcctggctgacgcattcatcctgatgcgctacccttttgagtccgccgaagctcagctgctgaacaagcagatttttgaaacaatctactacggggctctggaggcatcttgtgacctggccaaagaacagggaccctacgagacttatgaaggctcccctgtgtctaagggcatcctgcagtacgatatgtggaacgtcacaccaactgacctgtgggattggaaagtgctgaaggagaaaattgcaaagtatggcatccggaacagcctgctgatcgccccaatgcccactgcctctaccgctcagattctgggcaacaatgagtccatcgaaccatacacttctaacatctacacccggagagtcctgagcggggagttccagatcgtgaatccccacctgctgaaagacctgaccgaacggggactgtggcatgaggaaatgaagaaccagatcattgcctgcaatggcagtatccagtcaattcctgagatcccagacgatctgaaacagctgtacaagacagtctgggagatcagccagaaaactgtgctgaagatggcagccgaaagaggggctttcattgatcagtcacagagcctgaacatccacattgccgagcccaattacggaaagctgacctccatgcatttttatgggtggaaacagggactgaagactggcatgtactatctgcgcacccgaccagctgcaaaccccatccagtttaccctgaataaggagaaactgaaggacaaagaaaaggtgtccaaagaggaagaggaaaaggagagaaacacagccgctatggtgtgttctctggagaatagggatgaatgcctgatgtgtggcagtggaagcggagctactaacttcagcctgctgaagcaggctggagacgtggaggagaaccctggacctctgagtctgagggtcccactggcacctatcaccgatccacagcagctgcagctgagcccactgaaaggcctgagtctggtcgataaagagaacacaccacctgcactgagtggcactcgggtgctggcatcaaagaccgcccggagaattttccaggagccaaccgaacccaaaacaaaggccgctgcacctggggtcgaggacgaaccactgctgagagagaatcccaggcgcttcgtgatttttcctatcgaataccacgatatttggcagatgtataagaaagctgaggcaagtttctggacagctgaggaagtggacctgagcaaagacatccagcactgggaatccctgaagccagaggaaaggtacttcatttctcatgtgctggcattctttgccgctagtgacgggatcgtgaacgagaatctggtcgaacgctttagccaggaggtgcagatcactgaagcccgatgcttctatggatttcagattgctatggagaacatccattcagaaatgtacagcctgctgattgacacctatatcaaagatcctaaggagcgcgagttcctgtttaatgccattgagacaatgccatgtgtgaagaaaaaggcagactgggctctgcgatggatcggcgataaggaggctacttacggggaaagagtggtcgcattcgcagccgtggagggaattttcttttctggcagtttcgcttccatcttttggctgaaaaagcgaggcctgatgcctgggctgaccttttccaacgagctgatttctcgcgacgaaggcctgcactgcgatttcgcctgtctgatgtttaaacacctggtgcataagccctctgaggaacgagtccgggagatcattatcaacgcagtgaggatcgagcaggagttcctgacagaagccctgcctgtcaaactgattggcatgaattgcactctgatgaagcagtacatcgagtttgtggccgacaggctgatgctggaactgggattctcaaaggtgtttcgcgtcgagaacccattcgattttatggagaatatcagcctggaaggcaaaacaaacttctttgagaagagagtcggggaatatcagaggatgggcgtgatgagcagccccactgagaatagcttcaccctggacgccgatttttgagctagc

SEQ ID NO: 7 - skipped

SEQ ID NO: 8 - RNA, 2376 nt, synthetic construct
  source 1..2376: mol_type = other RNA
atgcacgtcatcaagagagacgggaggcaggaaagagtcatgttcgataaaatcacttcaagaatccagaaactgtgttacgggctgaacatggacttcgtcgatcctgcccagattaccatgaaagtgatccagggactgtactctggcgtcaccacagtggagctggacacactggccgctgaaaccgcagccacactgactaccaaacacccagattatgcaattctggctgcacggatcgccgtgagtaatctgcataaggagacaaagaaagtcttctcagacgtgatggaggacctgtacaattatatcaaccctcacaatgggaaacattcaccaatggtcgctaagagcactctggacattgtgctggccaacaaagatcggctgaacagcgctatcatctacgaccgggatttcagttacaactacttcggctttaagacactggagagatcatatctgctgaaaatcaatgggaaggtggccgaacggcctcagcacatgctgatgagagtcagcgtgggcattcataaggaggacattgatgccgctatcgaaacttacaacctgctgagcgagcgctggttcacccacgcttcccctacactgtttaacgcaggaaccaatcgaccacagctgagcagctgcttcctgctgagcatgaaggacgattccatcgagggcatctacgacaccctgaaacagtgcgcactgatttctaagagtgccggcgggatcggagtcgctgtgagttgtattcgggcaaccggctcatatatcgccggcacaaacggcaacagcaacgggctggtccccatgctgagggtgtacaacaatacagcccgctatgtggatcagggaggcaacaagagaccaggagcatttgccatctacctggaaccctggcacctggacattttcgagtttctggatctgaagaaaaatactggcaaagaggaacagagggctcgcgacctgttctttgcactgtggattcccgacctgttcatgaagagggtggagaccaaccaggactggagcctgatgtgccccaatgagtgtcctgggctggatgaagtgtggggagaggaatttgaaaaactgtacgccagttatgagaagcagggccgagtgcggaaagtggtcaaggcccagcagctgtggtacgctatcattgagagccagacagaaactggcaccccctacatgctgtataaagactcttgcaaccgcaagagtaaccagcagaatctggggaccatcaaatgcagcaatctgtgtacagagattgtggaatatacttccaaggatgaggtcgccgtgtgtaacctggcatcactggccctgaatatgtacgtcacaagcgagcacacttatgacttcaagaaactggctgaagtgaccaaagtggtcgtgaggaatctgaacaagatcattgacatcaactactatcccgtgcctgaggcctgcctgagcaataagagacataggcccatcgggattggagtgcagggcctggctgacgcattcatcctgatgcgctacccttttgagtccgccgaagctcagctgctgaacaagcagatttttgaaacaatctactacggggctctggaggcatcttgtgacctggccaaagaacagggaccctacgagacttatgaaggctcccctgtgtctaagggcatcctgcagtacgatatgtggaacgtcacaccaactgacctgtgggattggaaagtgctgaaggagaaaattgcaaagtatggcatccggaacagcctgctgatcgccccaatgcccactgcctctaccgctcagattctgggcaacaatgagtccatcgaaccatacacttctaacatctacacccggagagtcctgagcggggagttccagatcgtgaatccccacctgctgaaagacctgaccgaacggggactgtggcatgaggaaatgaagaaccagatcattgcctgcaatggcagtatccagtcaattcctgagatcccagacgatctgaaacagctgtacaagacagtctgggagatcagccagaaaactgtgctgaagatggcagccgaaagaggggctttcattgatcagtcacagagcctgaacatccacattgccgagcccaattacggaaagctgacctccatgcatttttatgggtggaaacagggactgaagactggcatgtactatctgcgcacccgaccagctgcaaaccccatccagtttaccctgaataaggagaaactgaaggacaaagaaaaggtgtccaaagaggaagaggaaaaggagagaaacacagccgctatggtgtgttctctggagaatagggatgaatgcctgatgtgtggcagt

SEQ ID NO: 9 - RNA, 57 nt, synthetic construct
  source 1..57: mol_type = other RNA
gctactaacttcagcctgctgaagcaggctggagacgtggaggagaaccctggacct

SEQ ID NO: 10-15 - skipped (6 entries)

SEQ ID NO: 16 - RNA, 1170 nt, synthetic construct
  source 1..1170: mol_type = other RNA
atgctgagtctgagggtcccactggcacctatcaccgatccacagcagctgcagctgagcccactgaaaggcctgagtctggtcgatgcagcggccacaccacctgcactgagtggcactcgggtgctggcatcaaagaccgcccggagaattttccaggagccaaccgaacccaaaacaaaggccgctgcacctggggtcgaggacgaaccactgctgagagagaatcccaggcgcttcgtgatttttcctatcgaataccacgatatttggcagatgtataagaaagctgaggcaagtttctggacagctgaggaagtggacctgagcaaagacatccagcactgggaatccctgaagccagaggaaaggtacttcatttctcatgtgctggcattctttgccgctagtgacgggatcgtgaacgagaatctggtcgaacgctttagccaggaggtgcagatcactgaagcccgatgcttctatggatttcagattgctatggagaacatccattcagaaatgtacagcctgctgattgacacctatatcaaagatcctaaggagcgcgagttcctgtttaatgccattgagacaatgccatgtgtgaagaaaaaggcagactgggctctgcgatggatcggcgataaggaggctacttacggggaaagagtggtcgcattcgcagccgtggagggaattttcttttctggcagtttcgcttccatcttttggctgaaaaagcgaggcctgatgcctgggctgaccttttccaacgagctgatttctcgcgacgaaggcctgcactgcgatttcgcctgtctgatgtttaaacacctggtgcataagccctctgaggaacgagtccgggagatcattatcaacgcagtgaggatcgagcaggagttcctgacagaagccctgcctgtcaaactgattggcatgaattgcactctgatgaagcagtacatcgagtttgtggccgacaggctgatgctggaactgggattctcaaaggtgtttcgcgtcgagaacccattcgattttatggagaatatcagcctggaaggcaaaacaaacttctttgagaagagagtcggggaatatcagaggatgggcgtgatgagcagccccactgagaatagcttcaccctggacgccgatttttga

SEQ ID NO: 17 - RNA, 1170 nt, synthetic construct
  source 1..1170: mol_type = other RNA
atgctgagtctgagggtcccactggcacctatcaccgatccacagcagctgcagctgagcccactgaaaggcctgagtctggtcgatgcagcgaacacaccacctgcactgagtggcactcgggtgctggcatcaaagaccgcccggagaattttccaggagccaaccgaacccaaaacaaaggccgctgcacctggggtcgaggacgaaccactgctgagagagaatcccaggcgcttcgtgatttttcctatcgaataccacgatatttggcagatgtataagaaagctgaggcaagtttctggacagctgaggaagtggacctgagcaaagacatccagcactgggaatccctgaagccagaggaaaggtacttcatttctcatgtgctggcattctttgccgctagtgacgggatcgtgaacgagaatctggtcgaacgctttagccaggaggtgcagatcactgaagcccgatgcttctatggatttcagattgctatggagaacatccattcagaaatgtacagcctgctgattgacacctatatcaaagatcctaaggagcgcgagttcctgtttaatgccattgagacaatgccatgtgtgaagaaaaaggcagactgggctctgcgatggatcggcgataaggaggctacttacggggaaagagtggtcgcattcgcagccgtggagggaattttcttttctggcagtttcgcttccatcttttggctgaaaaagcgaggcctgatgcctgggctgaccttttccaacgagctgatttctcgcgacgaaggcctgcactgcgatttcgcctgtctgatgtttaaacacctggtgcataagccctctgaggaacgagtccgggagatcattatcaacgcagtgaggatcgagcaggagttcctgacagaagccctgcctgtcaaactgattggcatgaattgcactctgatgaagcagtacatcgagtttgtggccgacaggctgatgctggaactgggattctcaaaggtgtttcgcgtcgagaacccattcgattttatggagaatatcagcctggaaggcaaaacaaacttctttgagaagagagtcggggaatatcagaggatgggcgtgatgagcagccccactgagaatagcttcaccctggacgccgatttttga

SEQ ID NO: 18 - RNA, 1170 nt, synthetic construct
  source 1..1170: mol_type = other RNA
atgctgagtctgagggtcccactggcacctatcaccgatccacagcagctgcagctgagcccactgaaaggcctgagtctggtcgataaagagaacgcaccacctgcactgagtggcactcgggtgctggcatcaaagaccgcccggagaattttccaggagccaaccgaacccaaaacaaaggccgctgcacctggggtcgaggacgaaccactgctgagagagaatcccaggcgcttcgtgatttttcctatcgaataccacgatatttggcagatgtataagaaagctgaggcaagtttctggacagctgaggaagtggacctgagcaaagacatccagcactgggaatccctgaagccagaggaaaggtacttcatttctcatgtgctggcattctttgccgctagtgacgggatcgtgaacgagaatctggtcgaacgctttagccaggaggtgcagatcactgaagcccgatgcttctatggatttcagattgctatggagaacatccattcagaaatgtacagcctgctgattgacacctatatcaaagatcctaaggagcgcgagttcctgtttaatgccattgagacaatgccatgtgtgaagaaaaaggcagactgggctctgcgatggatcggcgataaggaggctacttacggggaaagagtggtcgcattcgcagccgtggagggaattttcttttctggcagtttcgcttccatcttttggctgaaaaagcgaggcctgatgcctgggctgaccttttccaacgagctgatttctcgcgacgaaggcctgcactgcgatttcgcctgtctgatgtttaaacacctggtgcataagccctctgaggaacgagtccgggagatcattatcaacgcagtgaggatcgagcaggagttcctgacagaagccctgcctgtcaaactgattggcatgaattgcactctgatgaagcagtacatcgagtttgtggccgacaggctgatgctggaactgggattctcaaaggtgtttcgcgtcgagaacccattcgattttatggagaatatcagcctggaaggcaaaacaaacttctttgagaagagagtcggggaatatcagaggatgggcgtgatgagcagccccactgagaatagcttcaccctggacgccgatttttga

SEQ ID NO: 19 - RNA, 1170 nt, synthetic construct
  source 1..1170: mol_type = other RNA
atgctgagtctgagggtcccactggcacctatcaccgatccacagcagctgcagctgagcccactgaaaggcctgagtctggtcgataaagagaacacaccacctgcactgagtggcactcgggtgctggcatcaaagaccgccgcgagagctttccaggagccaaccgaacccaaaacaaaggccgctgcacctggggtcgaggacgaaccactgctgagagagaatcccaggcgcttcgtgatttttcctatcgaataccacgatatttggcagatgtataagaaagctgaggcaagtttctggacagctgaggaagtggacctgagcaaagacatccagcactgggaatccctgaagccagaggaaaggtacttcatttctcatgtgctggcattctttgccgctagtgacgggatcgtgaacgagaatctggtcgaacgctttagccaggaggtgcagatcactgaagcccgatgcttctatggatttcagattgctatggagaacatccattcagaaatgtacagcctgctgattgacacctatatcaaagatcctaaggagcgcgagttcctgtttaatgccattgagacaatgccatgtgtgaagaaaaaggcagactgggctctgcgatggatcggcgataaggaggctacttacggggaaagagtggtcgcattcgcagccgtggagggaattttcttttctggcagtttcgcttccatcttttggctgaaaaagcgaggcctgatgcctgggctgaccttttccaacgagctgatttctcgcgacgaaggcctgcactgcgatttcgcctgtctgatgtttaaacacctggtgcataagccctctgaggaacgagtccgggagatcattatcaacgcagtgaggatcgagcaggagttcctgacagaagccctgcctgtcaaactgattggcatgaattgcactctgatgaagcagtacatcgagtttgtggccgacaggctgatgctggaactgggattctcaaaggtgtttcgcgtcgagaacccattcgattttatggagaatatcagcctggaaggcaaaacaaacttctttgagaagagagtcggggaatatcagaggatgggcgtgatgagcagccccactgagaatagcttcaccctggacgccgatttttga

SEQ ID NO: 20 - RNA, 1170 nt, synthetic construct
  source 1..1170: mol_type = other RNA
atgctgagtctgagggtcccactggcacctatcaccgatccacagcagctgcagctgagcccactgaaaggcctgagtctggtcgatgcagcggccacaccacctgcactgagtggcactcgggtgctggcatcaaagaccgccgcgagagctttccaggagccaaccgaacccaaaacaaaggccgctgcacctggggtcgaggacgaaccactgctgagagagaatcccaggcgcttcgtgatttttcctatcgaataccacgatatttggcagatgtataagaaagctgaggcaagtttctggacagctgaggaagtggacctgagcaaagacatccagcactgggaatccctgaagccagaggaaaggtacttcatttctcatgtgctggcattctttgccgctagtgacgggatcgtgaacgagaatctggtcgaacgctttagccaggaggtgcagatcactgaagcccgatgcttctatggatttcagattgctatggagaacatccattcagaaatgtacagcctgctgattgacacctatatcaaagatcctaaggagcgcgagttcctgtttaatgccattgagacaatgccatgtgtgaagaaaaaggcagactgggctctgcgatggatcggcgataaggaggctacttacggggaaagagtggtcgcattcgcagccgtggagggaattttcttttctggcagtttcgcttccatcttttggctgaaaaagcgaggcctgatgcctgggctgaccttttccaacgagctgatttctcgcgacgaaggcctgcactgcgatttcgcctgtctgatgtttaaacacctggtgcataagccctctgaggaacgagtccgggagatcattatcaacgcagtgaggatcgagcaggagttcctgacagaagccctgcctgtcaaactgattggcatgaattgcactctgatgaagcagtacatcgagtttgtggccgacaggctgatgctggaactgggattctcaaaggtgtttcgcgtcgagaacccattcgattttatggagaatatcagcctggaaggcaaaacaaacttctttgagaagagagtcggggaatatcagaggatgggcgtgatgagcagccccactgagaatagcttcaccctggacgccgatttttga

SEQ ID NO: 21 - protein, 389 aa, synthetic construct
MLSLRVPLAPITDPQQLQLSPLKGLSLVDAAATPPALSGTRVLASKTARRIFQEPTEPKTKAAAPGVEDEPLLRENPRRFVIFPIEYHDIWQMYKKAEASFWTAEEVDLSKDIQHWESLKPEERYFISHVLAFFAASDGIVNENLVERFSQEVQITEARCFYGFQIAMENIHSEMYSLLIDTYIKDPKEREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEYQRMGVMSSPTENSFTLDADF

SEQ ID NO: 22 - protein, 389 aa, synthetic construct
MLSLRVPLAPITDPQQLQLSPLKGLSLVDAANTPPALSGTRVLASKTARRIFQEPTEPKTKAAAPGVEDEPLLRENPRRFVIFPIEYHDIWQMYKKAEASFWTAEEVDLSKDIQHWESLKPEERYFISHVLAFFAASDGIVNENLVERFSQEVQITEARCFYGFQIAMENIHSEMYSLLIDTYIKDPKEREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEYQRMGVMSSPTENSFTLDADF

SEQ ID NO: 23 - protein, 389 aa, synthetic construct
MLSLRVPLAPITDPQQLQLSPLKGLSLVDKENAPPALSGTRVLASKTARRIFQEPTEPKTKAAAPGVEDEPLLRENPRRFVIFPIEYHDIWQMYKKAEASFWTAEEVDLSKDIQHWESLKPEERYFISHVLAFFAASDGIVNENLVERFSQEVQITEARCFYGFQIAMENIHSEMYSLLIDTYIKDPKEREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEYQRMGVMSSPTENSFTLDADF

SEQ ID NO: 24 - protein, 389 aa, synthetic construct
MLSLRVPLAPITDPQQLQLSPLKGLSLVDKENTPPALSGTRVLASKTAARAFQEPTEPKTKAAAPGVEDEPLLRENPRRFVIFPIEYHDIWQMYKKAEASFWTAEEVDLSKDIQHWESLKPEERYFISHVLAFFAASDGIVNENLVERFSQEVQITEARCFYGFQIAMENIHSEMYSLLIDTYIKDPKEREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEYQRMGVMSSPTENSFTLDADF

SEQ ID NO: 25 - protein, 389 aa, synthetic construct
MLSLRVPLAPITDPQQLQLSPLKGLSLVDAAATPPALSGTRVLASKTAARAFQEPTEPKTKAAAPGVEDEPLLRENPRRFVIFPIEYHDIWQMYKKAEASFWTAEEVDLSKDIQHWESLKPEERYFISHVLAFFAASDGIVNENLVERFSQEVQITEARCFYGFQIAMENIHSEMYSLLIDTYIKDPKEREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEYQRMGVMSSPTENSFTLDADF

SEQ ID NO: 26 - DNA, 1165 bp, synthetic construct
  source 1..1165: mol_type = other DNA
tgagtctgagggtcccactggcacctatcaccgatccacagcagctgcagctgagcccactgaaaggcctgagtctggtcgatgcagcggccacaccacctgcactgagtggcactcgggtgctggcatcaaagaccgccgcgagagctttccaggagccaaccgaacccaaaacaaaggccgctgcacctggggtcgaggacgaaccactgctgagagagaatcccaggcgcttcgtgatttttcctatcgaataccacgatatttggcagatgtataagaaagctgaggcaagtttctggacagctgaggaagtggacctgagcaaagacatccagcactgggaatccctgaagccagaggaaaggtacttcatttctcatgtgctggcattctttgccgctagtgacgggatcgtgaacgagaatctggtcgaacgctttagccaggaggtgcagatcactgaagcccgatgcttctatggatttcagattgctatggagaacatccattcagaaatgtacagcctgctgattgacacctatatcaaagatcctaaggagcgcgagttcctgtttaatgccattgagacaatgccatgtgtgaagaaaaaggcagactgggctctgcgatggatcggcgataaggaggctacttacggggaaagagtggtcgcattcgcagccgtggagggaattttcttttctggcagtttcgcttccatcttttggctgaaaaagcgaggcctgatgcctgggctgaccttttccaacgagctgatttctcgcgacgaaggcctgcactgcgatttcgcctgtctgatgtttaaacacctggtgcataagccctctgaggaacgagtccgggagatcattatcaacgcagtgaggatcgagcaggagttcctgacagaagccctgcctgtcaaactgattggcatgaattgcactctgatgaagcagtacatcgagtttgtggccgacaggctgatgctggaactgggattctcaaaggtgtttcgcgtcgagaacccattcgattttatggagaatatcagcctggaaggcaaaacaaacttctttgagaagagagtcggggaatatcagaggatgggcgtgatgagcagccccactgagaatagcttcaccctggacgccgatttttg